(19) United States
(12) Patent Application Publication
(10) Pub. No.: US 2004/0101818 A1
(43) Pub. Date: May 27, 2004

(54) Gene expression profiles associated with osteoblast differentiation

(76) Inventors: Darren Ji, Cincinnati, OH (US); Douglas W. Axelrod, Cincinnati, OH (US); Jonathan S. Cook, Cincinnati, OH (US); Neelam Jaiswal, Cincinnati, OH (US); Richard Einstein, Gaithersburg, MD (US); Adam Houghton, Cincinnati, OH (US); Lawrence Mertz, Gaithersburg, MD (US)

Correspondence Address: MORGAN LEWIS & BOCKIUS LLP, 1111 PENNSYLVANIA AVENUE NW, WASHINGTON, DC, 20004, US

(21) Appl. No.: 10/450,826

(22) PCT Filed: Dec. 18, 2001
(86) PCT No.: PCT/US01/48276

(51) Int. Cl. C12Q 1/68; C12Q 1/00

(52) U.S. Cl. 435/4; 435/6

(57) ABSTRACT

The present invention identifies genes whose expression pattern is altered when precursor stem cells undergo differentiation into osteoblasts. The genes identified may be used as markers for the differentiation process. The present invention also provides methods to screen agents that are capable of modulating the differentiation process. The present invention also provides methods of identifying therapeutic agents that stimulate bone information by analyzing the expression of one or more of the genes identified.

### RELATED APPLICATONS

This application claims the benefit of U.S. Provisional Applications 60/255,882 (filed Dec. 18, 2000) and 60/285,691 (filed Apr. 24, 2001), all of which are herein incorporated by reference in their entirety.

### BACKGROUND OF THE INVENTION

Bone is a dynamic tissue in which old tissue is broken down and new tissue is synthesized. Control of the rate of breakdown and synthesis of new bone tissue is critical to the integrity of the skeletal structure. When the rates become unbalanced, serious conditions may result.

The process of synthesizing new bone tissue is mediated by osteoblasts. During the process of synthesizing new bone tissue, osteoblasts differentiate from precursor stem cells to mature bone-forming cells. During this differentiation, numerous genes undergo changes in expression levels. The expression levels of various enzymes and structural proteins, for example alkaline phosphatase and Type-1 collagen, are up-regulated while other genes are down-regulated.

In order to treat a condition characterized by an imbalance in the rates of breakdown and synthesis of bone tissue, it may be desirable to increase or decrease the rate of break down and/or synthesis. Thus, in a number of clinical applications, it may desirable to enhance the rate of bone formation by promoting the differentiation of precursor stem cells into osteoblasts. One application which is particularly important is the treatment of osteoporosis which is characterized by a decrease in bone mass making the bones more fragile and subject to fracture. Other potential uses for reagents capable of affecting the synthesis of bone tissue include the healing of broken bones, recovery after surgical procedures involving bones and the like.

While the changes in the expression levels of a number of individual genes have been identified, the investigation of the global changes in gene expression which occur in precursor stem cells as they differentiate into osteoblasts has not been reported. Such information would be useful, for example, in assessing the effects of a course of treatment designed to change the rate of formation of bone tissue. Accordingly, there exists a need for the investigation of the changes in global gene expression levels as well as the need for the identification of new molecular markers associated with the differentiation of precursor stem cells into osteoblasts. Furthermore, identification of additional genes involved in differentiation may allow development of reagents designed to alter their expression levels and thereby allow control of the differentiation process. In addition, identification of the genes involved in the process allows their use as diagnostic or prognostic markers which are uniquely associated with differentiation.

### SUMMARY OF THE INVENTION

The present invention relates to the elucidation of the global changes in gene expression in precursor stem cells undergoing the process of differentiation into osteoblasts. In one aspect, the present invention relates to detecting a change in an expression level of one or more genes or gene families associated with the differentiation of one or more precursor stem cells into one or more osteoblasts. In a related aspect, the activity of a protein encoded by a gene or member of a gene family may be assayed. Such assays may be conducted by themselves or in conjunction with determining an expression level. In some aspects, it may be desirable to determine an expression level of one or more genes or members of a gene family in Table 1 while at the same time determining an activity level of one or more proteins encoded by a gene or member of a gene family of Table 1. The genes or member of gene families for which expression levels are determined may be the same or different as the genes encoding the proteins assayed. Thus, in some embodiments, it may be desirable to determine the expression level of a gene and the activity level of the protein encoded by the gene. In other embodiments, it may be desirable to determine the expression level of one gene while determining the activity level of a protein encoded by another gene. Those skilled in the art will appreciate that the expression and/or activity level of any number of genes and proteins may be determined according to the present invention.

In a related aspect, the present invention includes methods of screening for an agent that modulates the differentiation of a precursor stem cell into an osteoblast, comprising: preparing a first gene or gene family expression profile and/or assaying for an activity of a protein encoded by a gene or member of a gene family of Table 1 in a cell population comprising one or more precursor stem cells; contacting the cell population with an agent; preparing a second gene or gene family expression profile and/or assaying for an activity of a protein encoded by a gene or member of a gene family of Tables 1 or 2 of the cell population after being contacted with the agent; and comparing the first and second expression profiles and/or activities.

In one aspect, the present invention provides a method of diagnosing a condition characterized by abnormal deposition of bone tissue, comprising detecting the level of expression in a tissue sample of one or more genes or gene families from Table 1 and/or assaying for an activity of a protein encoded by a gene or member of a gene family of Table 1, wherein differential expression and/or activity is indicative of inadequate bone tissue deposition.

In another aspect, the present invention also includes methods of monitoring the treatment of a patient with a condition characterized by abnormal bone tissue deposition, comprising administering a pharmaceutical composition to the patient, preparing a gene or gene family expression profile and/or assaying for an activity of a protein encoded by a gene or member of a gene family of Table 1 from a cell or tissue sample from the patient and comparing the patient expression profile and/or activity to an expression profile and/or activity from a precursor stem cell population or an osteoblast cell population.

In another aspect, the present invention also includes methods of treating a patient with a condition characterized by abnormal bone tissue deposition, comprising administering a pharmaceutical composition to the patient; preparing a gene or gene family expression profile and/or assaying for an activity of a protein encoded by a gene or member of a gene family of Table 1 from a cell or tissue sample from the patient comprising precursor stem cells; and comparing the patient expression profile and/or activity to an expression profile and/or activity from an untreated cell population comprising precursor stem cells.

The invention includes methods of diagnosing a condition characterized by an abnormal rate of formation of osteoblasts in a patient comprising detecting the level of expression in a tissue sample of one or more genes or gene families from Table 1 and/or assaying for an activity of a protein encoded by a gene or member of a gene family of Table 1; wherein differential expression and/or activity is indicative of an abnormal rate of formation of osteoblasts.

The invention includes a method of monitoring the treatment of a patient with a condition characterized by abnormal rate of formation of osteoblasts, comprising administering a pharmaceutical composition to the patient, preparing a gene or gene family expression profile and/or assaying for an activity of a protein encoded by a gene or member of a gene family of Table 1 from a cell or tissue sample from the patient and comparing the patient expression profile and/or activity to an expression profile and/or activity from a precursor stem cell population or an osteoblast cell population.

In a related aspect, the present invention provides a method of treating a patient with a condition characterized by an abnormal rate of formation of osteoblasts, comprising administering to the patient a pharmaceutical composition, wherein the composition alters the expression of at least one gene or gene family in Table 1 and/or alters an activity of a protein encoded by a gene or member of a gene family of Table 1, preparing an expression profile and/or assaying for an activity from a cell or tissue sample from the patient comprising precursor stem cells and comparing the patient expression profile and/or activity to an expression profile and/or activity from an untreated cell population comprising precursor stem cells.

The invention further includes a method of diagnosing osteoporosis in a patient comprising detecting the level of expression in a tissue sample of one or more genes or gene families from Table 1 and/or assaying for an activity of a protein encoded by a gene or member of a gene family of Table 1; wherein differential expression and/or activity is indicative of osteoporosis.

In a related aspect, the present invention provides a method of monitoring the treatment of a patient with osteoporosis, comprising administering a pharmaceutical composition to the patient, preparing a gene or gene family expression profile and/or assaying for an activity of a protein encoded by a gene or member of a gene family of Table 1 from a cell or tissue sample from the patient and comparing the patient expression profile and/or activity to an expression profile and/or activity from a precursor stem cell population or an osteoblast cell population.

In one aspect, the present invention provides a method of treating a patient with osteoporosis, comprising administering to the patient a pharmaceutical composition, wherein the composition alters the expression of at least one gene or gene family in Table 1 and/or alters an activity of a protein encoded by a gene or member of a gene family of Table 1, preparing an expression profile and/or assaying for an activity of a protein encoded by a gene or member of a gene family of Table 1 from a cell or tissue sample from the patient comprising precursor stem cells and comparing the patient expression profile and/or activity to an expression profile and/or activity from an untreated cell population comprising precursor stem cells.

Also included in the inventions are methods of screening for an agent capable of ameliorating the effects of osteoporosis, comprising exposing a cell to the agent; and detecting the expression level of one or more genes or gene families from Table 1 and/or assaying for an activity of a protein encoded by a gene or member of a gene family of Table 1.

In one aspect, the present invention is a method of monitoring the progression of bone tissue deposition in a patient, comprising detecting the level of expression in a tissue sample of one or more genes or gene families from Table 1 and/or assaying for an activity of a protein encoded by a gene or member of a gene family of Table 1; wherein differential expression and/or activity is indicative of bone tissue deposition.

In a related aspect, the present invention is a method of screening for an agent capable of modulating the deposition of bone tissue, comprising exposing a cell to the agent and detecting the expression level of one or more genes or gene families from Table 1 and/or assaying for an activity of a protein encoded by a gene or member of a gene family of Table 1.

All of these methods may include the step of detecting the expression levels of at least 1, 2, 3, 4, 5, 6, 7, 8, 9, 10 or more genes or members of the gene families in Table 1. Preferably, expression of all of the genes or members of the gene families or nearly all of the genes or members of the gene families in Table 1 may be detected. In a related aspect, the methods of the present invention may comprise the step of assaying for an activity of at least 1, 2, 3, 4, 5, 6, 7, 8, 9, 10 or more proteins encoded by a gene or member of a gene family of Table 1. In some preferred embodiments, the methods of the present invention may comprise both determining an expression level of one or more genes of members of a gene family of Table 1 and assaying an activity of one or more proteins encoded by a gene or member of a gene family of Table 1. In some embodiments, the expression level of a gene and the activity level of the protein encoded by the same gene may be determined. In other embodiments, the expression level of at least one gene may be determined while the activity level of at least one protein encoded by a different gene may be determined.

In one aspect, the present invention provides a method for identifying an agent that modulates the differentiation of precursor stem cells into osteoblasts comprising contacting a cell population with the agent and assaying for at least one activity of at least one gene or the activity of at least one member of a gene family identified in Table 1. In a related aspect, the present invention provides a method of monitoring the treatment of a patient with a condition characterized by abnormal bone deposition comprising administering a pharmaceutical composition to the patient and assaying for at least one activity of at least one gene or one member of a gene family identified in Table 1. The present invention also includes a method of diagnosing a condition characterized by the abnormal rate of formation of osteoblast comprising detecting the level of activity of at least one gene or one member of a gene family identified in Table 1.

In some preferred aspects, the present invention encompasses a composition comprising at least two oligonucleotides, wherein each of the oligonucleotides comprises a sequence that specifically hybridizes to one or more genes or members of a gene family in Table 1. In some aspects, the composition may comprise at least 1, 2, 3, 4, 5, 6, 7, 8, 9, 10 oligonucleotides, wherein each of the oligonucleotides comprises a sequence that specifically hybridizes to one or more genes or members of a gene family in Table 1. In some embodiments, one or more of the oligonucleotides may be attached to a solid support. The solid support may be any known to those skilled in the art including, but not limited to, a membrane, a glass support, a filter, a tissue culture dish, a polymeric material and a silicon support.

In a preferred aspect, the present invention provides a solid support to which is attached at least two oligonucleotides, wherein each of the oligonucleotides comprises a sequence that specifically hybridizes to at least one gene or to at least one member of a gene family in Table 1. In some embodiments, at least one oligonucleotide is attached covalently to the solid support. In some embodiments, at least one oligonucleotide is attached non-covalently to the solid support. Oligonucleotides may be attached to the solid supports of the invention at any density known to those skilled in the art, for example, at about at least 10 different oligonucleotides in discrete locations per square centimeter, at about at least 100 different oligonucleotides in discrete locations per square centimeter, at about at least 1000 different oligonucleotides in discrete locations per square centimeter and/or at about at least 10,000 different oligonucleotides in discrete locations per square centimeter. The selection of an appropriate density for a given application is a routine procedure for those skilled in the art.

The invention also includes computer systems comprising a database containing information identifying the expression level of one or more members of one or more of the gene families in Table 1 and/or the activity level of one or more proteins encoded by a gene or by a member of a gene family of Table 1 in a resting precursor stem cell and/or a precursor stem cell differentiating into an osteoblast and/or an osteoblast; and a user interface to view the information. The database may further comprise sequence information for one or more of the genes of one or one or more members of one or more of the gene families of Table 1. The database may comprise information identifying the expression level for one or more genes or one or more members of one or more of the gene families in the set of gene families expressed in a precursor stem cell that is not differentiating. The database may comprise information identifying the expression level for one or more genes or one or more members of one or more of the gene families in the set of genes or gene families expressed in a precursor stem cell that is differentiating into a cell type other than an osteoblast. The database may comprise information identifying the expression level for one or more genes or one or more members of one or more of the gene families in the set of genes or gene families expressed in a precursor stem cell that is differentiating into an osteoblast. The database may further contain or be linked to descriptive information from an external database, which information correlates said genes and/or gene families to records in the external database.

Lastly the invention includes methods of using the disclosed computer systems to present information identifying the expression level in a tissue or cell of a set of genes and/or gene families comprising at least one of the genes or gene families in Table 1, comprising comparing the expression level of at least one gene or gene family in Table 1 in the tissue or cell to the level of expression of the gene in the database. The invention also includes methods of using the disclosed computer systems to present information identifying the activity level in a tissue or cell of one or more proteins encoded by one or more genes and/or members of a gene family comprising at least one of the genes or gene families in Table 1, comprising comparing the activity level of at least one protein encoded by one gene or member of a gene family in Table 1 in the tissue or cell to the level of activity of the protein in the database.

### BRIEF DESCRIPTION OF THE DRAWINGS

FIG. 1A shows the expression level of an RNA related to aquaporin mRNA as a function of time in the absence (control-open circles solid line) and in the presence (BMP-2-open squares dashed line) of 300 ng/ml BMP-2. FIG. 1B shows the expression level of the RNA as a function of time in the absence (control-open circles solid line) and in the presence (open triangles dashed line) of 1 ng/ml TGFb-1.

FIG. 2A shows the expression level of an RNA related to the mRNA encoding Mpv 17 protein as a function of time in the absence (control-open circles solid line) and in the presence (open squares dashed line) of 300 ng/ml BMP-2. FIG. 2B shows the expression level as a function of time in the absence (control-open circles solid line) and in the presence (open triangles dashed line) of 1 ng/ml TGFb-1.

FIG. 3A shows the expression level of an RNA related to claudin protein mRNA as a function of time in the absence (control-open circles) and in the presence (open squares dashed line) of 300 ng/ml BMP-2. FIG. 3B shows the expression level as a function of time in the absence (control-open circles) and in the presence (open triangles dashed line) of 1 ng/ml TGFb-1.

FIG. 4A shows the expression level of an RNA related to SM22α mRNA as a function of time in the absence (control-open circles) and in the presence (open squares dashed line) of 300 ng/ml BMP-2. FIG. 4B shows the expression level as a function of time in the absence (control-open circles) and in the presence (open triangles dashed line) of 1 ng/ml TGFb-1.

FIG. 5 shows the expression level of the RNA of EST: AA722810 as a function of time in the absence (control-open circles solid line) and in the presence (open triangles dashed line) of 1 ng/ml TGFb-1.

FIG. 6A shows the expression level of the RNA related to the mRNA encoding PEDF as a function of time in the absence (control-open circles solid line) and in the presence (open squares dashed line) of 300 ng/ml BMP-2. FIG. 6B shows the expression level as a function of time in the absence (control-open circles solid line) and in the presence (open triangles dashed line) of 1 ng/ml TGFb-1.

FIG. 7A shows the expression level of TGFb II receptor mRNA as a function of time in the absence (control-open circles, solid line) and the presence (BMP-2-open squares, dashed line) of 300 ng/ml BMP-2. FIG. 7B shows the expression level of the RNA as a function of time in the absence (control-open circles, solid line) and in the presence (open triangles, dashed line) of 1 ng/ml TGFb-1.

FIG. 8 shows the expression level of Bradykinin B2 Receptor mRNA as a function of time in the absence (control-open circles, solid line) and the presence (BMP-2-open squares, dashed line) of 300 ng/ml BMP-2.

FIG. 9 shows the expression level of an mRNA related to Frizzled-related protein frpHE as a function of time in the absence (control-open circles, solid line) and in the presence (open triangles, dashed line) of 1 ng/ml TGFb-1.

FIG. 10A shows the expression level of AH Receptor mRNA as a function of time in the absence (control-open circles, solid line) and the presence (BMP-2-open squares, dashed line) of 300 ng/ml BMP-2. FIG. 10B shows the expression level of the RNA as a function of time in the absence (control-open circles, solid line) and in the presence (open triangles, dashed line) of 1 ng/ml TGFb-1.

FIG. 11A shows the expression level of GPx-4 mRNA as a function of time in the absence (control-open circles, solid line) and the presence (BMP-2-open squares, dashed line) of 300 ng/ml BMP-2. FIG. 11B shows the expression level of the RNA as a function of time in the absence (control-open circles, solid line) and in the presence (open triangles, dashed line) of 1 ng/ml TGFb-1.

FIG. 12A shows the expression level of Preproenkephalin mRNA as a function of time in the absence (control-open circles, solid line) and the presence (BMP-2-open squares, dashed line) of 300 ng/ml BMP-2. FIG. 12B shows the expression level of the RNA as a function of time in the absence (control-open circles, solid line) and in the presence (open triangles, dashed line) of 1 ng/ml TGFb-1.

FIG. 13 shows the expression level of Cartilage Derived Morphogenic Protein mRNA as a function of time in the absence (control-open circles, solid line) and the presence (open triangles, dashed line) of 1 ng/ml TGFb-1.

FIG. 14 shows the expression level of the RNA related to aquaporin mRNA as a function of time in the absence (control-open circles) and in the presence (BMP-2-closed squares) of 300 ng/ml BMP-2 or in the presence, (TGFb-1-closed circles) of 1 ng/ml TGFb-1.

FIG. 15 shows the expression level of the RNA related to C1 inhibitor mRNA as a function of time in the absence (control-open circles) and in the presence (13 MP-2-closed squares) of 300 ng/ml BMP-2 or in the presence (TGFb-1-closed circles) of 1 ng/ml TGFb-1.

FIG. 16 shows the expression level of RNA related to claudin 11 mRNA as a function of time in the absence (control-open circles) and in the presence (BMP-2-closed squares) of 300 ng/ml BMP-2 or in the presence (TGFb-1-closed circles) of 1 ng/ml TGF-β1.

FIG. 17 shows the expression level of DKK-1 mRNA as a function of time in the absence (control-open circles) and in the presence (BMP-2-closed squares) of 300 ng/ml BMP-2 or in the presence (TGFb-1-closed circles) of 1 ng/ml TGFb-1.

FIG. 18 shows the expression level of ESTAI869864 RNA as a function of time in the absence (control-open circles) and in the presence (BMP-2-closed squares) of 300 ng/ml BMP-2 or in the presence (TGFb-1-closed circles) of 1 ng/ml TGFb-1.

FIG. 19 shows the expression level of the RNA related to stromal cell derived receptor-1α mRNA as a function of time in the absence (control-open circles) and in the presence (BMP-2-closed squares) of 300 ng/ml BMP-2 or in the presence (TGFb-1-closed circles) of 1 ng/ml TGFb-1.

FIG. 20 shows the expression level of TGFb II Receptor mRNA as a function of time in the absence (control-solid circles) and in the presence of 300 ng/ml of BMP-2 (closed triangle, dotted line) or in the presence of 1 ng/ml TGFb1 (open square, solid line) in the case for HFSCs. For HMSCs, the mRNA was measured as a function of time in the absence (control-solid circle, solid line) and in the presence of 300 ng/ml of BMP-2 (solid triangle, dotted line) or in the presence of either 1 ng/ml TGFb (open square, solid line) or 100 nM dexamethasone (crosses, solid line).

FIG. 21 shows the expression level of Bradykinin B2 Receptor mRNA as a function of time in the absence (control-solid circles) and in the presence of 300 ng/ml of BMP-2 (closed triangle, dotted line) or in the presence of 1 ng/ml TGFb 1 (open square, solid line) in the case for HFSCs. For HMSCs, the mRNA was measured as a function of time in the absence (control-solid circle, solid line) and in the presence of 300 ng/ml of BMP-2 (solid triangle, dotted line) or in the presence of either 1 ng/ml TGFb (open square, solid line) or 100 nM dexamethasone (crosses, solid line).

FIG. 22 shows the expression level of the mRNA related to Frizzled related protein frpHE as a function of time in the absence (control-solid circles) and in the presence of 300 ng/ml of BMP-2 (closed triangle, dotted line) or in the presence of 1 ng/ml TGFb1 (open square, solid line) in the case for HFSCs. For HMSCs, the mRNA was measured as a function of time in the absence (control-solid circle, solid line) and in the presence of 300 ng/ml of BMP-2 (solid triangle, dotted line) or in the presence of either 1 ng/ml TGFb (open square, solid line) or 100 nM dexamethasone (crosses, solid line).

FIG. 23 shows the expression level of AH Receptor mRNA as a function of time in the absence (control-solid circles) and in the presence of 300 ng/ml of BMP-2 (closed triangle, dotted line) or in the presence of 1 ng/ml TGFb1 (open square, solid line) in the case for HFSCs. For HMSCs, the mRNA was measured as a function of time in the absence (control-solid circle, solid line) and in the presence of 300 ng/ml of BMP2 (solid triangle, dotted line) or in the presence of either 1 ng/ml TGFb (open square, solid line) or 100 nM dexamethasone (crosses, solid line).

FIG. 24 shows the expression level of GPx-4 mRNA as a function of time in the absence (control-solid circles) and in the presence of 300 ng/ml of BMP-2 (closed triangle, dotted line) or in the presence of 1 ng/ml TGFb1 (open square, solid line) in the case for HFSCs. For HMSCs, the mRNA was measured as a function of time in the absence (control-solid circle, solid line) and in the presence of 300 ng/ml of BMP-2 (solid triangle, dotted line) or in the presence of either 1 ng/ml TGFb (open square, solid line) or 100 nM dexamethasone (crosses, solid line).

FIG. 25 shows the expression level of preproenkephalin mRNA as a function of time in the absence (control-solid circles) and in the presence of 300 ng/ml of BMP-2 (closed triangle, dotted line) or in the presence of 1 ng/ml TGFb1 (open square, solid line) in the case for HFSCs. For HMSCs, the mRNA was measured as a function of time in the absence (control-solid circle, solid line) and in the presence of 300 ng/ml of BMP-2 (solid triangle, dotted line) or in the presence of either 1 ng/ml TGFb (open square, solid line) or 100 nM dexamethasone (crosses, solid line).

FIG. 26 shows the expression level of Cartilage-derived morphogenic protein mRNA as a function of time in the absence (control-solid circles) and in the presence of 300 ng/ml of BMP-2 (closed triangle, dotted line) or in the presence of 1 ng/ml TGFb1 (open square, solid line) in the case for HFSCs. For HMSCs, the mRNA was measured as a function of time in the absence (control-solid circle, solid line) and in the presence of 300 ng/ml of BMP-2 (solid triangle, dotted line) or in the presence of either 1 ng/ml TGFb (open square, solid line) or 100 nM dexamethasone (crosses, solid line).

### DETAILED DESCRIPTION

Many biological functions are accomplished by altering the expression of various genes through transcriptional (e.g. through control of initiation, provision of RNA precursors, RNA processing, etc.) and/or translational control. For example, fundamental biological processes such as cell cycle, cell differentiation and cell death, are often characterized by the variations in the expression levels of groups of genes.

Changes in gene expression also are associated with pathogenesis. For example, the lack of sufficient expression of functional tumor suppressor genes and/or the over expression of oncogene/protooncogenes could lead to tumorgenesis or hyperplastic growth of cells (Marshall, (1991) Cell 64:313-326; Weinberg, (1991) Science 254:1138-1146). Thus, changes in the expression levels of particular genes (e.g., oncogenes or tumor suppressors) serve as signposts for the presence and progression of various diseases.

Monitoring changes in gene expression may also provide certain advantages during drug screening development. Often drugs are screened for the ability to interact with a major target without regard to other effects the drugs have on cells. Often such other effects cause toxicity in the whole animal, which prevent the development and use of the potential drug.

The present inventors have examined cell populations comprising precursor stem cells and cell populations comprising precursor stem cells that have been induced to differentiate into osteoblasts to identify the global changes in gene expression during this differentiation process. These global changes in gene expression, also referred to as expression profiles, provide useful markers for diagnostic uses as well as markers that can be used to monitor disease states, disease progression, toxicity, drug efficacy and drug metabolism.

The expression profiles have been used to identify individual genes that are differentially expressed under one or more conditions. In addition, the present invention identifies families of genes that are differentially expressed. As used herein, “gene families” includes, but is not limited to, the specific genes identified by accession number herein, as well as related sequences. Related sequences may be, for example, sequences having a high degree of sequence identity with a specifically identified sequence either at the nucleotide level or at the level of amino acids of the encoded polypeptide. A high degree of sequence identity is seen to be at least about 65% sequence identity at the nucleotide level to said genes, preferably about 80 or 85% sequence identity or more preferably about 90 or 95% or more sequence identity to said genes. With regard to amino acid identity of encoded polypeptides, a high degree of identity is seen to be at least about 50% identity, more preferably about 75% identity and most preferably about 85% or more sequence identity. In particular, related sequences include homologous genes from different organisms. For example, if the specifically identified gene is from a non-human mammal, the gene family would encompass homologous genes from other mammals including humans. If the specifically identified gene is a human gene, gene family would encompass the homologous gene from different organisms. Those skilled in the art will appreciate that a homologous gene may be of different length and may comprise regions with differing amounts of sequence identity to a specifically identified sequence.

Assay Formats

The genes and sequences identified as being differentially expressed in the cell population induced to differentiate as well as related sequences may be used in a variety of nucleic acid detection assays to detect or quantititate the expression level of a gene or multiple genes in a given sample. For example, traditional Northern blotting, nuclease protection, RT-PCR, QPCR (quantitative RT-PCR), Taqman® and differential display methods may be used for detecting gene expression levels. Those methods are useful for some embodiments of the invention. However, methods and assays of the invention are most efficiently designed with hybridization-based methods for detecting the expression of a large number of genes.

Any hybridization assay format may be used, including solution-based and solid support-based assay formats. Solid supports containing oligonucleotide probes for differentially expressed genes of the invention can be filters, polyvinyl chloride dishes, silicon or glass based chips, etc. Such supports and hybridization methods are widely available, for example, those disclosed by WO 95/11755. Any solid surface to which oligonucleotides can be bound, either directly or indirectly, either covalently or non-covalently, can be used. A preferred solid support is a high density array or DNA chip. These contain a particular oligonucleotide probe in a predetermined location on the array. Each predetermined location may contain more than one molecule of the probe, but each molecule within the predetermined location has an identical sequence. Such predetermined locations are termed features. There may be, for example, from 2, 10, 100, 1000 to 10,000, 100,000 or 400,000 of such features on a single solid support. The solid support, or the area within which the probes are attached may be any convenient size and may preferably be on the order of a square centimeter.

Oligonucleotide probe arrays for expression monitoring can be made and used according to any techniques known in the art (see for example, Lockhart et al., (1996) Nat. Biotech. 14, 1675-1680; McGall et al., (1996) Proc. Nat. Acad. Sci. USA 93, 13555-13460). Such probe arrays may contain at least two or more oligonucleotides that are complementary to or hybridize to two or more of the genes described in Table 1. For instance, such arrays may also contain oligonucleotides that are complementary or hybridize to at least about 1, 2, 3, 4, 5, 6, 7, 8, 9, 10, 20, 30, 50, 70 or more the genes described herein.

The genes which are assayed according to the present invention are typically in the form of mRNA or reverse transcribed mRNA. The genes may be cloned or not. The genes may be amplified or not. The cloning itself does not appear to bias the representation of genes within a population. However, it may be preferable to use polyadenylated RNA as a source, as it can be used with less processing steps.

Table 1 provides the Accession numbers and name for the sequences of the differentially expressed markers (SEQ ID NO: 1-60). The sequences of the genes in GenBank are expressly incorporated herein.

Probes based on the sequences of the genes described above may be prepared by any commonly available method. Oligonucleotide probes for interrogating the tissue or cell sample are preferably of sufficient length to specifically hybridize only to appropriate, complementary genes or transcripts. Typically the oligonucleotide probes will be at least 10, 12, 14, 16, 18, 20 or 25 nucleotides in length. In some cases longer probes of at least 30, 40 or 50 nucleotides will be desirable.

As used herein, oligonucleotide sequences that are complementary to one or more of the genes and/or gene families described in Table 1, refer to oligonucleotides that are capable of hybridizing under stringent conditions to at least part of the nucleotide sequences of said genes. Such hybridizable oligonucleotides will typically exhibit at least about 75% sequence identity at the nucleotide level to said genes, preferably about 80 or 85% sequence identity or more preferably about 90 or 95% or more sequence identity to said genes.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals may also be produced by intrinsic fluorescence of the array components themselves. A single background signal can be calculated for the entire array, or a different background signal may be calculated for each target nucleic acid. In a preferred embodiment, background is calculated as the average hybridization signal intensity for the lowest 5 to 10% of the probes in the array, or, where a different background signal is calculated for each target gene, for the lowest 5 to 10% of the probes for each gene. Of course, one of skill in the art will appreciate that where the probes to a particular gene hybridize well and thus appear to be specifically binding to a target sequence, they should not be used in a background signal calculation. Alternatively, background may be calculated as the average hybridization signal intensity produced by hybridization to probes that are not complementary to any sequence found in the sample (e.g., probes directed to nucleic acids of the opposite sense or to genes not found in the sample such as bacterial genes where the sample is mammalian nucleic acids). Background can also be calculated as the average signal intensity produced by regions of the array that lack any probes at all.

The phrase “hybridizing specifically to” refers to the binding, duplexing, or hybridizing of a molecule substantially to or only to a particular nucleotide sequence or sequences under stringent conditions when that sequence is present in a complex mixture (e.g., total cellular) DNA or RNA.

Assays and methods of the invention may utilize available formats to simultaneously screen at least about 100, preferably about 1000, more preferably about 10,000 and most preferably about 1,000,000 different nucleic acid hybridizations.

The terms “mismatch control” or “mismatch probe” refer to a probe whose sequence is deliberately selected not to be perfectly complementary to a particular target sequence. For each mismatch (MM) control in a high-density array there typically exists a corresponding perfect match (PM) probe that is perfectly complementary to the same particular target sequence. The mismatch may comprise one or more bases.

While the mismatch(s) may be located anywhere in the mismatch probe, terminal mismatches are less desirable as a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions.

The term “perfect match probe” refers to a probe that has a sequence that is perfectly complementary to a particular target sequence. The test probe is typically perfectly complementary to a portion (subsequence) of the target sequence. The perfect match (PM) probe can be a “test probe” or a “normalization control” probe, an expression level control probe and the like. A perfect match control or perfect match probe is, however, distinguished from a “mismatch control” or “mismatch probe” as defined herein.

As used herein a “probe” is defined as a nucleic acid, capable of binding to a target nucleic acid of complementary sequence through one or more types of chemical bonds, usually through complementary base pairing, usually through hydrogen bond formation. As used herein, a probe may include natural (i.e., A, G, U, C or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

The term “stringent conditions” refers to conditions under which a probe will hybridize to its target subsequence, but with only insubstantial hybridization to other sequences or to other sequences such that the difference may be identified. Stringent conditions are sequence-dependent and will be different in different circumstances. Longer sequences hybridize specifically at higher temperatures. Generally, stringent conditions are selected to be about 5° C. lower than the thermal melting point (Tₘ) for the specific sequence at a defined ionic strength and pH.

Typically, stringent conditions will be those in which the salt concentration is at least about 0.01 to 1.0 M sodium ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide.

The “percentage of sequence identity” or “sequence identity” is determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences. The percentage is calculated by determining the number of positions at which the identical residue (e.g., nucleic acid base or amino acid residue) occurs in both sequences to yield the number of matched positions, dividing the number of matched positions by the total number of positions in the window of comparison and multiplying the result by 100 to yield the percentage of sequence identity.

Percentage sequence identity can be calculated by the local homology algorithm of Smith & Waterman, (1981) Adv. Appl. Math. 2:482-485; by the homology alignment algorithm of Needleman & Wunsch, (1970) J. Mol. Biol. 48:443-445; or by computerized implementations of these algorithms (GAP & BESTFIT in the GCG Wisconsin Software Package, Genetics Computer Group) or by manual alignment and visual inspection.

Percentage sequence identity when calculated using the programs GAP or BESTFIT is calculated using default gap weights. The BESTFIT program has two alignment variables, the gap creation penalty and the gap extension penalty, which can be modified to alter the stringency of a nucleotide and/or amino acid alignment produced by the program. Parameter values used in the percent identity determination were default values previously established for version 8.0 of BESTFIT (see Dayhoff, (1979) Atlas of Protein Sequence and Structure, National Biomedical Research Foundation, pp. 353-358).

Probe Design

One of skill in the art will appreciate that an enormous number of array designs are suitable for the practice of this invention. In some preferred embodiments, a high density array may be used. The high density array will typically include a number of probes that specifically hybridize to the sequences of interest (see WO 99/32660 for methods of producing probes for a given gene or genes). In addition, in a preferred embodiment, the array will include one or more control probes.

High density array chips of the invention include “test probes” as defined herein. Test probes could be oligonucleotides that range from about 5 to about 45 or 5 to about 500 nucleotides, more preferably from about 10 to about 40 nucleotides and most preferably from about 15 to about 40 nucleotides in length. In other particularly preferred embodiments, the probes are 20 or 25 nucleotides in length. In another preferred embodiment, test probes are double or single strand nucleic acid sequences, preferably DNA sequences. Nucleic acid sequences may be isolated or cloned from natural sources or amplified from natural sources using native nucleic acid as templates. These probes have sequences complementary to particular subsequences of the genes whose expression they are designed to detect. Thus, the test probes are capable of specifically hybridizing to the target nucleic acid they are to detect.

In addition to test probes that bind the target nucleic acid(s) of interest, the high density array can contain a number of control probes. The control probes fall into three categories referred to herein as (1) normalization controls; (2) expression level controls; and (3) mismatch controls.

Normalization controls are oligonucleotide or other nucleic acid probes that are complementary to labeled reference oligonucleotides or other nucleic acid sequences that are added to the nucleic acid sample to be screened. The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g., fluorescence intensity) read from all other probes in the array are divided by the signal (e.g., fluorescence intensity) from the control probes thereby normalizing the measurements.

Virtually any probe may serve as a normalization control. However, it is recognized that hybridization efficiency varies with base composition and probe length. Preferred normalization probes are selected to reflect the average length of the other probes present in the array, however, they can be selected to cover a range of lengths. The normalization control(s) can also be selected to reflect the (average) base composition of the other probes in the array, however in a preferred embodiment, only one or a few probes are used and they are selected such that they hybridize well (i.e., no secondary structure) and do not match any target-specific probes.

Expression level controls are probes that hybridize specifically with constitutively expressed genes in the biological sample. Virtually any constitutively expressed gene provides a suitable target for expression level controls. Typically expression level control probes have sequences complementary to subsequences of constitutively expressed “housekeeping genes” including, but not limited to the actin gene, the transferrin receptor gene, the GAPDH gene, and the like.

Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). Preferred mismatch probes contain a central mismatch. Thus, for example, where a probe is a twenty-mer, a corresponding mismatch probe will have the identical sequence except for a single base mismatch (e.g., substituting a G, C or T for an A) at any of positions six through fourteen (the central mismatch).

Mismatch probes thus provide a control for non-specific binding or cross hybridization to a nucleic acid in the sample other than the target to which the probe is directed. Mismatch probes also indicate whether a hybridization is specific or not.

For example, if the target is present the perfect match probes should be consistently brighter than the mismatch probes. In addition, if all central mismatches are present, the mismatch probes can be used to detect a mutation. The difference in intensity between the perfect match and the mismatch probe provides a good measure of the concentration of the hybridized material.

Nucleic Acid Samples

As is apparent to one of ordinary skill in the art, nucleic acid samples, which may be DNA and/or RNA, used in the methods and assays of the invention may be prepared by any available method or process. Methods of isolating total mRNA are well known to those of skill in the art. For example, methods of isolation and purification of nucleic acids are described in detail in Chapter 3 of Tijssen, (1993) Laboratory Techniques in Biochemistry and Molecular Biology: Hybridization With Nucleic Acid Probes, Elsevier Press. Such samples include RNA samples, but also include cDNA synthesized from a mRNA sample isolated from a cell or tissue of interest. Such samples also include DNA amplified from the cDNA, and RNA transcribed from the amplified DNA. One of skill in the art would appreciate that it is desirable to inhibit or destroy RNase present in homogenates before homogenates can be used.

Biological samples may be of any biological tissue or fluid or cells from any organism as well as cells raised in vitro, such as cell lines and tissue culture cells. Frequently, the sample will be a “clinical sample” which is a sample derived from a patient. Typical clinical samples include, but are not limited to, sputum, blood, blood-cells (e.g., white cells), tissue or fine needle biopsy samples, urine, peritoneal fluid, and pleural fluid, or cells therefrom.

Biological samples may also include sections of tissues, such as frozen sections or formalin fixed sections taken for histological purposes.

Forming High Density Arrays

Methods of forming high density arrays of oligonucleotides with a minimal number of synthetic steps are known. The oligonucleotide analogue array can be synthesized on a solid substrate by a variety of methods, including, but not limited to, light-directed chemical coupling, and mechanically directed coupling (see U.S. Pat. No. 5,143,854).

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in WO 93/09668. High density nucleic acid arrays can also be fabricated by depositing premade or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

Hybridization

Nucleic acid hybridization simply involves contacting a probe and target nucleic acid under conditions where the probe and its complementary target can form stable hybrid duplexes through complementary base pairing (see WO 99/32660). The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA or RNA:DNA) will form even where the annealed sequences are not perfectly complementary. Thus specificity of hybridization is reduced at lower stringency. Conversely, at higher stringency (e.g., higher temperature and/or lower salt and/or in the presence of destabilizing reagents) successful hybridization tolerates fewer mismatches. One of skill in the art will appreciate that hybridization conditions may be selected to provide any degree of stringency. In a preferred embodiment, hybridization is performed at low stringency in this case in 6×SSPE-T at 37° C. (0.005% Triton x-100) to ensure hybridization and then subsequent washes are performed at higher stringency (e.g., 1×SSPE-T at 37° C.) to eliminate mismatched hybrid duplexes. Successive washes may be performed at increasingly higher stringency (e.g., down to as low as 0.25×SSPET at 37° C. to 50° C.) until a desired level of hybridization specificity is obtained. Stringency can also be increased by addition of destabilizing agents such as formamide. Hybridization specificity may be evaluated by comparison of hybridization to the test probes with hybridization to the various controls that can be present (e.g., expression level control, normalization control, mismatch controls, etc.).

In general, there is a trade-off between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

Signal Detection

The hybridized nucleic acids are typically detected by detecting one or more labels attached to the sample nucleic acids. The labels may be incorporated by any of a number of means well known to those of skill in the art (see WO 99/32660).

Databases

The present invention includes relational databases containing sequence information, for instance, for the genes and members of the gene families of Table 1 as well as gene expression information in various tissue samples saved on computer readable medium and/or a user interface. Databases may also contain information associated with a given sequence or tissue sample such as descriptive information about the gene associated with the sequence information, or descriptive information concerning the clinical status of the tissue sample, or the patient from which the sample was derived. The database may be designed to include different parts, for instance a sequence database and a gene expression database. Methods for the configuration and construction of such databases are widely available, for instance, see U.S. Pat. No. 5,953,727, which is herein incorporated by reference in its entirety.

The databases of the invention may be linked to an outside or external database. In a preferred embodiment, the external database is GenBank and the associated databases maintained by the National Center for Biotechnology Information (NCBI).

Any appropriate computer platform may be used to perform the necessary comparisons between sequence information, gene expression information and any other information in the database or provided as an input. For example, a large number of computer workstations are available from a variety of manufacturers, such has those available from Silicon Graphics. Client/server environments, database servers and networks are also widely available and appropriate platforms for the databases of the invention.

The databases of the invention may be used to produce, among other things, electronic Northerns that allow the user to determine the cell type or tissue in which a given gene is expressed and to allow determination of the abundance or expression level of a given gene in a particular tissue or cell.

The databases of the invention may also be used to present information identifying the expression level in a sample of a set of genes comprising one or more of the sequences of genes or members of the gene families of Table 1, comprising comparing the expression level of at least one gene or member of a gene family of Table 1 in the sample to the level of expression of the gene in the database. Such methods may be used to predict the differentiation state of the precursor stem cells present in a given sample by comparing the level of expression of a gene or member of a gene family in Table 1 from a sample to the expression levels found in normal, un-differentiated precursor stem cells and/or precursor stem cells induced to differentiate into osteoblasts and/or precursor stem cells induced to differentiate into a cell type other than an osteoblast and/or osteoblasts. Such methods may also be used in the drug or agent screening assays as described below.

Diagnostic uses for the Differentiation Markers

As described above, the genes and gene expression information provided in Table 1 may be used as diagnostic markers for the prediction or identification of the differentiation state of a sample comprising precursor stem cells. For instance, a tissue sample may be assayed by any of the methods described above, and the expression levels from a gene or member of a gene family from Table 1 may be compared to the expression levels found in undifferentiated precursor stem cells and/or precursor stem cells induced to differentiate into osteoblasts and/or precursor stem cells induced to differentiate into a cell type other than an osteoblast and/or osteoblasts. The comparison of expression data, as well as available sequence or other information may be done by researcher or diagnostician or may be done with the aid of a computer and databases as described above. Such methods may be used to diagnose or identify conditions characterized by abnormal bone deposition, reabsorption and/or abnormal rates of osteoblast differentiation.

Those skilled in the art will appreciate that a wide variety of conditions are associated with abnormal bone deposition or loss. Such conditions include, but are not limited to, osteoporosis, osteopenia, osteodystrophy, and various other osteopathic conditions. The methods of the present invention will be particularly useful in diagnosing or monitoring the treatment of conditions such as postmenopausal osteoporosis (PMO), glucocorticoid-induced osteoporosis (GIO) and male osteoporosis. Agents which modulate the expression of one or more the genes or gene families identified in Table 1 and/or modulate the activity of one or more of the proteins encoded by one or more of the genes or members of a gene family identified in Table 1 will be useful in treatment of the conditions.

In some preferred embodiments, the present invention may be used to diagnose and/or monitor the treatment of drug-induced abnormalities in bone formation or loss. For example, at present a combination of cyclosporine with prednisone is given to patients who have received an organ transplant in order to suppress tissue rejection. The combination causes rapid bone loss in a manner different than that observed with prednisone alone (such as elevated level of serum osteocalcin and 1,25(OH)₂-Vitamin D in patients treated with cyclosporine but not in patients treated with prednisone). Other drugs are also known to effect bone formation or loss. The anticonvulsant drugs diphenylhydantoin, phenobarbital and carbamazepine, and combination of these drugs, cause alterations in calcium metabolism. A decrease in bone density is observed in patients taking anticonvulsant drugs. Although heparin is an effective therapy for thromboembolic disorders, increased incidences of osteoporotic fractures have been reported in patients with heparin therapy hence the present invention will be useful to monitor patients undergoing heparin treatment.

Other embodiments of the present invention allow the diagnosis and/or monitoring of the treatment of other conditions that involve altered bone metabolism. For example, idiopathic juvenile osteoporosis (IJO) is a generalized decrease in mineralized bone in the absence of rickets or excessive bone resorption and typically occurs in children before the onset of puberty. In addition, thyroid diseases have been linked bone loss. A decrease in bone mass has been shown in patients with thyrotoxicosis causing these individuals to be at increased risk of having fractures. These individuals also sustain fractures at an earlier age than individuals who have never been thyrotoxic.

Other conditions in which the present invention will be useful include multiple myeloma and leukemia. Nearly 60% of patients with multiple myelomas have bone fractures with focal and lytic bone lesions and osteosclereotic bone lesions. Leukemia may also be associated with diffuse osteopenia and vertebral fracture in patients with acute lymphoblastic leukemia.

Another situation in which the present invention will be useful is the diagnosis and/or monitoring of the treatment of skeletal disease linked to breast cancer. Breast cancer frequently metastasizes to the skeleton and about 70% of patients with advanced cancer develop symptomatic skeletal disease. Moreover, the anticancer treatments presently in use have been shown to lead to early menopause and bone loss when given to premenopausal women.

The present invention will be useful in diagnosing and/or monitoring the treatment of chronic anemia associated with abnormal bone formation or loss. Homozygous beta-thalassemia is usually described as an example of chronic anemia predisposing to osteoporosis. Patients with thalassemia have expansion of bone marrow space with thinning of the adjacent trabeculae.

The present invention will be useful in diagnosing and/or monitoring the treatment of mastocytosis. Skeletal symptoms (osteopenia and vertebral fracture) are present in 60 to 75% of the patients with systemic mast cell disease.

Other conditions in which the present invention will find application are: Fanconi syndrome where osteomalacia is a common feature; fibrous dysplasia, McCune-Albright syndrome refers to patients with fibrous dysplasia with a sporadic, developmental disorder characterized by a unifocal or multifocal expanding fibrous lesion of bone-forming mesenchyme that often results in pain, fracture or deformity; osteogenesis imperfecta (OI, also called brittle bone disease) is associated with recurrent fractures and skeletal deformity, various skeletal dysplasias, i.e., osteochondroplasia which is characterized by abnormal development of cartilage and/or bone and other diseases such as achodroplasia, mucopolysacchaidoses, dysostosis and ischemic bone diseases.

The present invention will be particularly useful by providing one or markers which may be used as markers of bone turnover to determine osteoporosis.

The present invention may also be used in in vitro assays or treatments as a marker of osteoblast differentiation and/or proliferation.

The agents of the present invention may be used for a variety of purposes. In a preferred embodiment, they may be used in fracture repair of all types, i.e., non-union fractures, spinal fusion, accelerated healing of all types fractures from minor greenstick or compression fractures to comminuted, complicated fractures. Both local administrations to these fractures as well as parenteral administration which increases cartilage and bone formation, increases bone mass, and increases bone strength rapidly may be used. Another preferred embodiment of the present invention is the use of bone formation modulating agents in periodontal disease and/or for increasing bone around teeth.

Use of the Differentiation Markers for Monitoring Disease Progression

As described above, the genes and gene expression information provided in Table 1 may also be used as markers for the monitoring of disease progression, such as osteoporosis. For instance, a tissue sample may be assayed by any of the methods described above, and the expression levels for a gene or member of a gene family from Table 1 may be compared to the expression levels found in un-differentiated precursor stem cells and/or precursor stem cells induced to differentiate into osteoblasts and/or precursor stem cells induced to differentiate into a cell type other than an osteoblast and/or osteoblasts. The comparison of the expression data, as well as available sequence or other information may be done by researcher or diagnostician or may be done with the aid of a computer and databases as described above.

The markers of the invention may also be used to track or predict the progress or efficacy of a treatment regime in a patient. For instance, a patient's progress or response to a give drug may be monitored by creating a gene expression profile from a tissue or cell sample after treatment or administration of the drug. The gene expression profile may then be compared to a gene expression profile prepared from un-differentiated precursor stem cells and/or precursor stem cells induced to differentiate into osteoblasts and/or precursor stem cells induced to differentiate into a cell type other than an osteoblast and/or osteoblasts and/or from tissue or cells from the same patient before treatment. The gene expression profile may be made from at least one gene, preferably more than one gene, and most preferably all or nearly all of the genes in Table 1.

Use of the Differentiation Markers for Drug Screening

According to the present invention, the genes identified in Table 1 may be used as markers to evaluate the effects of a candidate drug or agent on a cell. A candidate drug or agent can be screened for the ability to stimulate the transcription or expression of a given marker or markers or to down-regulate or counteract the transcription or expression of a marker or markers. For instance, agents that modulate, induce or inhibit gene expression in a sample to that which resembles a gene expression profile in an osteoblast differentiated cell population may be screened for the ability to modulate the differentiation process, bone depositions, etc. According to the present invention one can also compare the specificity of a drug effect by looking at the number of markers which the drug has and comparing them. More specific drugs will have less transcriptional targets. Similar sets of markers identified for two drugs indicates a similarity of effects.

Assays to monitor the expression of a marker or markers as defined in Table 1 may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

In one assay format, gene chips containing probes to one or more genes or members of a gene family from Table 1 may be used to directly monitor or detect changes in gene expression in the treated or exposed cell as described in more detail above. In another format, cell lines that contain reporter gene fusions between the open reading frame and/or 5′→3′ regulatory regions of a gene or member of a gene family in Table 1 and any assayable fusion partner may be prepared. Numerous assayable fusion partners are known and readily available including the firefly luciferase gene and the gene encoding chloramphenicol acetyltransferase (Alam et al., (1990) Anal. Biochem. 188:245-254). Cell lines containing the reporter gene fusions are then exposed to the agent to be tested under appropriate conditions and time. Differential expression of the reporter gene between samples exposed to the agent and control samples identifies agents which modulate the expression of the nucleic acid.

Additional assay formats may be used to monitor the ability of the agent to modulate the expression of a gene or member of a gene family identified in Table 1. For instance, as described above, mRNA expression may be monitored directly by hybridization of probes to the nucleic acids of the invention. Cell lines are exposed to the agent to be tested under appropriate conditions and time and total RNA or mRNA is isolated by standard procedures such those disclosed in Sambrook et al., (1989) Molecular Cloning: A Laboratory Manual, Cold Spring Harbor Laboratory Press.

In another assay format, cells or cell lines are first identified which express the gene products of the invention physiologically. Cells and/or cell lines so identified would be expected to comprise the necessary cellular machinery such that the fidelity of modulation of the transcriptional apparatus is maintained with regard to exogenous contact of agent with appropriate surface transduction mechanisms and/or the cytosolic cascades. Such cell lines may be, but are not required to be, bone marrow derived. Further, such cells or cell lines may be transduced or transfected with an expression vehicle (e.g., a plasmid or viral vector) construct comprising an operable non-translated 5′-promoter containing end of the structural gene encoding the instant gene products fused to one or more antigenic fragments, which are peculiar to the instant gene products, wherein said fragments are under the transcriptional control of said promoter and are expressed as polypeptides whose molecular weight can be distinguished from the naturally occurring polypeptides or may further comprise an immunologically distinct tag or some other detectable marker or tag. Such a process is well known in the art (see Sambrook et al., (1989) Molecular Cloning: A Laboratory Manual, Cold Spring Harbor Laboratory Press).

Cells or cell lines transduced or transfected as outlined above are then contacted with agents under appropriate conditions; for example, the agent comprises a pharmaceutically acceptable excipient and is contacted with cells comprised in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37° C. Said conditions may be modulated as deemed necessary by one of skill in the art. Subsequent to contacting the cells with the agent, said cells are disrupted and the polypeptides of the lysate are fractionated such that a polypeptide fraction is pooled and contacted with an antibody to be further processed by immunological assay (e.g., ELISA, immunoprecipitation or Western blot). The pool of proteins isolated from the “agent-contacted” sample is then compared with a control sample where only the excipient is contacted with the cells and an increase or decrease in the immunologically generated signal from the “agent-contacted” sample compared to the control is used to distinguish the effectiveness of the agent.

Another embodiment of the present invention provides methods for identifying agents that modulate the levels or at least one activity of a protein(s) encoded by the genes in Table 1. Such methods or assays may utilize any means of monitoring or detecting the desired activity.

In one format, the relative amounts of a protein of the invention between a cell population that has been exposed to the agent to be tested compared to an unexposed control cell population may be assayed. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe, such as a specific antibody.

Agents that are assayed in the above methods can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site.

The agents of the present invention can be, as examples, peptides, small molecules, vitamin derivatives, as well as carbohydrates. Dominant negative proteins, DNA encoding these proteins, antibodies to these proteins, peptide fragments of these proteins or mimics of these proteins may be introduced into cells to affect function. “Mimic” used herein refers to the modification of a region or several regions of a peptide molecule to provide a structure chemically different from the parent peptide but topographically and functionally similar to the parent peptide (see Meyers, (1995) Molecular Biology and Biotechnology, VCH Publishers, 659-664). A skilled artisan can readily recognize that there is no limit as to the structural nature of the agents of the present invention.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXAMPLES

### Example 1

Identification of Genes Differentially Expressed in Differentiating Precursor Stem Cells

Human Fetal Stromal Cells (HFSCs) were obtained from Dr. Xu Cao, Department of Pathology at the University of Alabama These cells were isolated from the bone marrow of a twenty-week human embryo. BFSCs are derived from a primary culture and represent a heterogeneous population of osteoprogenitor cells. HFSCs exhibit a high replicative capacity, with a doubling time of approximately twenty hours. HFSCs retain a spindle-shaped morphology and have a uniform attachment throughout subcultivation. HFSCs can be sub-cultured up to twelve passages while retaining both proliferative and osteogenic capability.

HFSCs used for READS analysis or QPCR (quantitative RT-PCR) were cultured in Dulbecco's Modified Eagle Medium (DMEM)-high glucose or DMEM-low glucose supplemented with 10% Fetal Bovine Serum, respectively, at 37° C. in a humidified atmosphere containing 95% air and 5% CO₂ in the absence and presence of the indicated treatment. RNA was extracted from the cells at zero minutes, three hour, six hours, twelve hours, twenty-four hours, forty-eight hours, three days, six days, twelve days and twenty-four days. When indicated, cells were contacted with either bone morphogenic protein-2 (BMP-2) at 300 ng/ml or transforming growth factor beta (TGF-beta) at 1 ng/ml or cycloheximide at 1 μM. Cells were incubated for the period of time indicated and harvested.

Total cellular RNA was prepared from the human fetal stromal cells described above. Synthesis of cDNA was performed as previously described in WO 97/05286 and in Prashar et al., (1996) Proc. Natl. Acad. Sci. USA 93:659-663 (READs). Briefly, cDNA was synthesized according to the protocol described in the GibcoBRL kit for cDNA synthesis. The reaction mixture for first-strand synthesis included 6 μg of total RNA, and 200 ng of a mixture of one-base anchored oligo(dT) primers with all three possible anchored bases (ACGTAATACGACTCACTATAGGGCGAATTGGGTCGACTTTTTTTTTTTTTTTTT n1 (SEQ ID NO: 61) wherein n1=A/C or G) along with other components for first-strand synthesis reaction except reverse transcriptase. This mixture was incubated at 65° C. for five minutes, chilled on ice and the process repeated. Alternatively, the reaction mixture may include 10 μg of total RNA and 2 pmol of one of the two-base anchored. Oligo(dT) primers annealed such as RP5.0 (CTCTCAAGGATCTTACCGCTT₁₈AT) (SEQ ID NO: 62) or RP6.0 (TAATACCGCGCCACATAGCAT₁₈CG) (SEQ ID NO: 63) or RP9.2 (CAGGGTAGACGACGCTACGCT₁₈GA) (SEQ ID NO: 64) along with other components for first-strand synthesis reaction except reverse transcriptase. This mixture was then layered with mineral oil and incubated at 65° C. for seven minutes followed by 50° C. for another seven minutes. At this stage, 2 μl of Superscript® reverse transcriptase (200 units/μl; Gibco/BRL) was added quickly and mixed, and the reaction continued for one hour at 45-50° C. Second-strand synthesis was performed at 16° C. for two hours. At the end of the reaction, the cDNA was precipitated with ethanol and the yield of cDNA was calculated. In our experiments, 200 ng of cDNA was obtained from 10 μg of total RNA.

The adapter oligonucleotide sequences were A1 (TAGCGTCCGGCGCAGCGACGGCCAG (SEQ ID NO: 65) and A2 (GATCCTGGCCGTCGGCTGTCTGTCGGCGC) (SEQ ID NO: 66). One microgram of oligonucleotide A2 was first phosphorylated at the 5′ end using T4 polynucleotide kinase (PNK). After phosphorylation, PNK was heated denatured, and 1 μg of the oligonucleotide A1 was added along with 10× annealing buffer (1 M NaCl/100 mM Tris-HCl (pH 8.0), 10 mM EDTA (H 8.0)) in a final volume of 20 μl. This mixture was then heated at 65° C. for ten minutes followed by slow cooling to room temperature for thirty minutes, resulting in formation of the Y adapter at a final concentration of 100 ng/μl. About 20 ng of the cDNA was digested with 4 units of Bgl II in a final volume of 10:1 for thirty minutes at 37° C. Two microliters (4 ng of digested cDNA) of this reaction mixture was then used for ligation to 100 ng (fifty-fold) of the Y-shaped adapter in a final volume of 5 μl for sixteen hours at 15° C. After ligation, the reaction mixture was diluted with water to a final volume of 80 μl (adapter ligated cDNA concentration, 50 pg/μl) and heated at 65° C. for ten minutes to denature T4 DNA ligase and 2 μl aliquots (with 100 pg of cDNA) were used for PCR.

The following sets of primers were used for PCR amplification of the adapter ligated 3′-end cDNA: GAAGCCGAGACGTCGGTCG(T)₁₈ n1, n2 (SEQ ID NO: 67) (wherein n1, n2=AA, AC, AG, AT, CA, CC, CG, CT, GA, GC, GG or GT) as the 3′ primer with A1 as the 5′ primer or alternatively P5.0, RP6.0 or RP9.2 used as 3′ primers with primer A1.1 serving as the 5′ primer. To detect the PCR products on the display gel, 24 pmol of oligonucleotide A1 or A1.1 was 5′-end-labeled using 15:1 of [gamma-³²P]ATP (Amersham; 3000 Ci/mmol) and PNK in a final volume of 20 μl for thirty minutes at 37° C. After heat denaturing PNK at 65° C. for twenty minutes, the labeled oligonucleotide was diluted to a final concentration of 2 μM in 80 μl with unlabeled oligonucleotide A1.1. The PCR mixture (20 μl) consisted of 2 μl (100 pg) of the template, 2 μl of 10×PCR buffer (100 mM Tris.HCl (pH 8.3), 500 mM KCl), 2 μl of 15 mM MgCl₂ to yield 1.5 mM final Mg²⁺ concentration optimum in the reaction mixture, 200 μM dNTPs, 200 nM each 5′ and 3′ PCR primers, and 1 unit of Amplitaq® Gold. Primers and dNTPs were added after preheating the reaction mixture containing the rest of the components at 85° C. This “hot start” PCR was done to avoid amplification artifacts arising out of arbitrary annealing of PCR primers at lower temperature during transition from room temperature to 94° C. in the first PCR cycle. PCR consisted of five cycles of 94° C. for thirty seconds, 55° C. for two minutes, and 72° C. for sixty seconds followed by twenty-five cycles of 94° C. for thirty seconds, 60° C. for two minutes, and 72° C. for sixty seconds. A higher number of cycles resulted in smeary gel patterns. PCR products (2.5 μl) were analyzed on 6% polyacrylamide sequencing gel. For double or multiple digestion following adapter ligation, 13.2 μl of the ligated cDNA sample was 0.25 digested with a secondary restriction enzyme(s) in a final volume of 20 μl. From this solution, 3 μl was used as template for PCR. This template volume of 3 μl carried 100 pg of the cDNA and 10 mM MgCl₂ (from the 10× enzyme buffer), which diluted to the optimum of 1.5 mM in the final PCR volume of 20 μl. Since Mg²⁺ comes from the restriction enzyme buffer, it was not included in the reaction mixture when amplifying secondarily cut cDNA. Individual cDNA fragments corresponding to mRNA species were separated by denaturing polyacrylamide gel electrophoresis and visualized by autoradiography.

Bands identified as having different expression levels in treated versus untreated human fetal stromal cells were extracted from the display gels as described by Liang et al., (1995) Curr. Opin. Immunol. 7:274-280), reamplified using the 5′- and 3′-primers, and subcloned into pCR-Script with high efficiency using the PCR-Script cloning kit from Stratagene. Plasmids were sequenced by cycle sequencing on an ABI automated sequencer. Alternatively, bands were extracted (cored) from the display gels, PCR amplified and sequenced directly without subcloning.

The sequences thus identified are listed in Table 1 along with any related sequences as indicated by the designation “Related To” under the Class column in Table 1. This table also provides the GenBank accession number and name of the genes related to the sequences identified by the READs analysis. The identity column of Table 1 contains information on the closeness of the sequence determined by READS analysis to the sequence in the public database. For example, the first entry of Table 1 indicates that the sequence of the fragment identified by READS is identical to the published sequence in 343 of the 348 positions of the READS fragment and has 98% sequence identity to the published sequence. The last column of Table 1 also indicates whether the expression of the sequence identified by READS analysis was up- or down-regulated in the differentiation process.

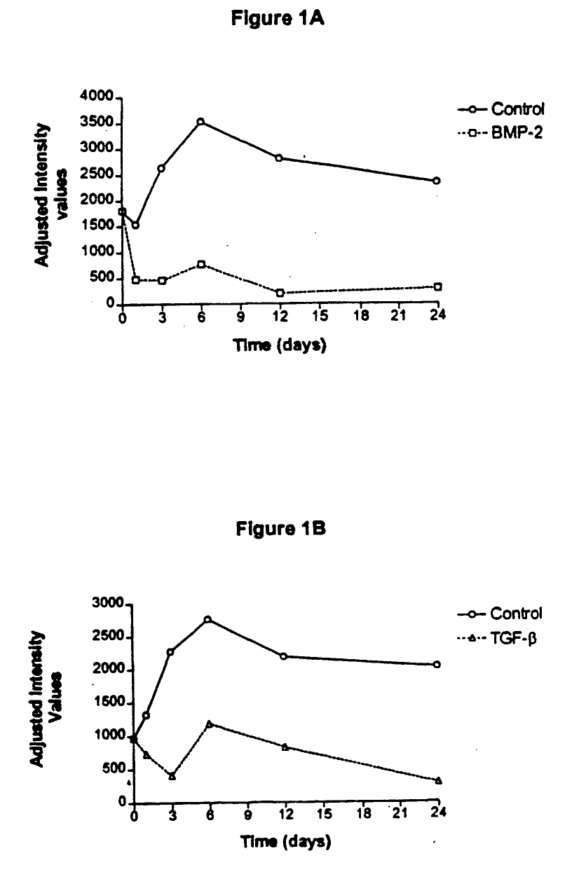

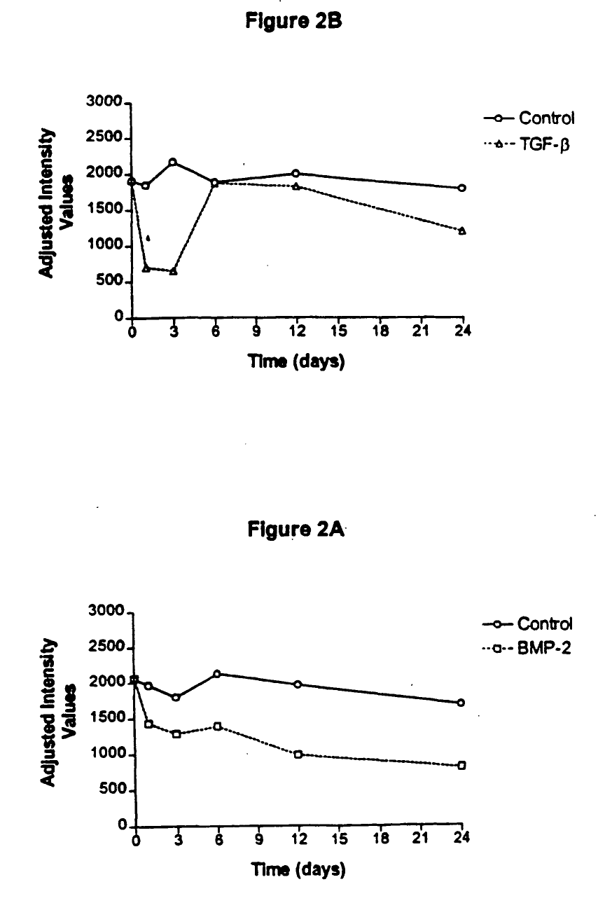

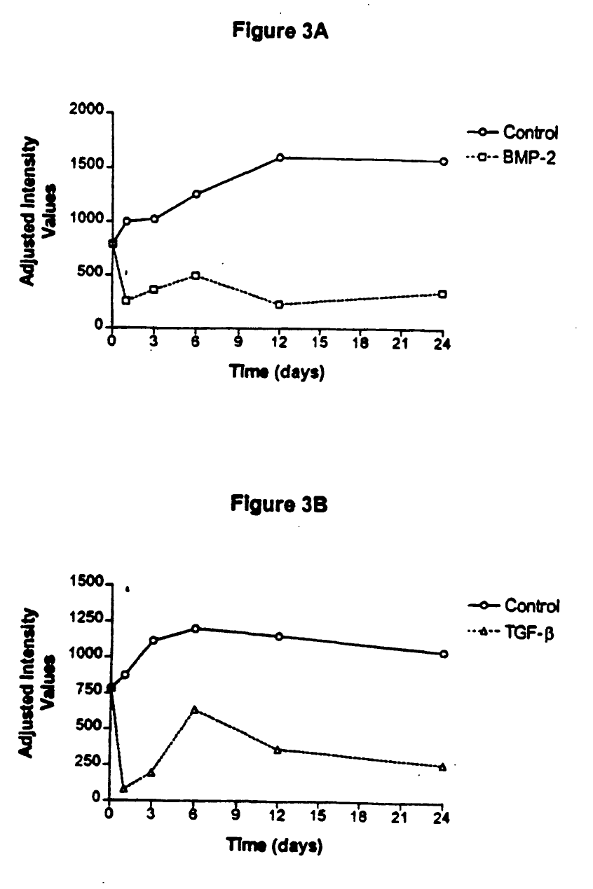

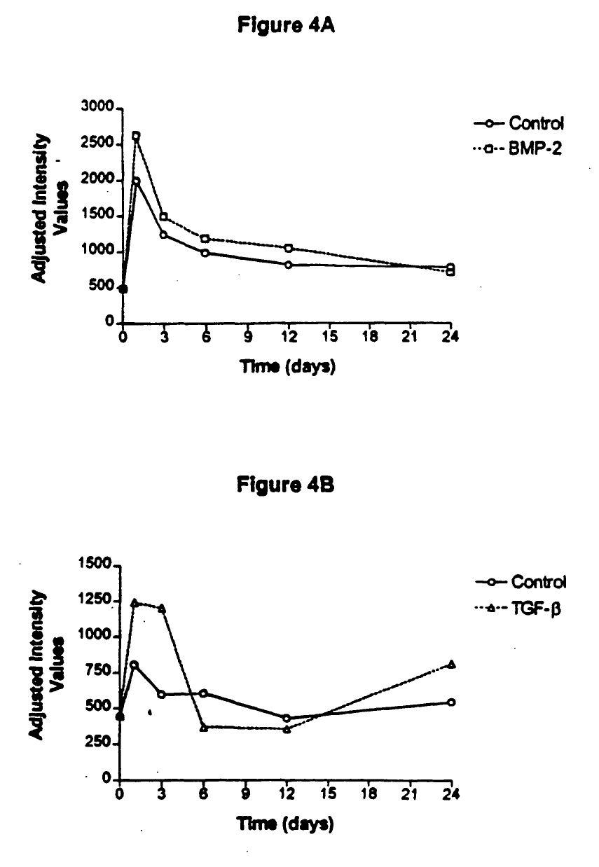

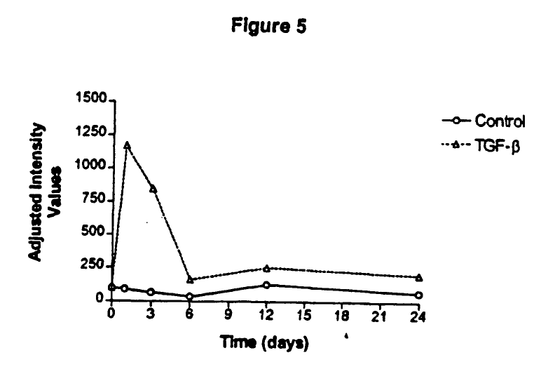

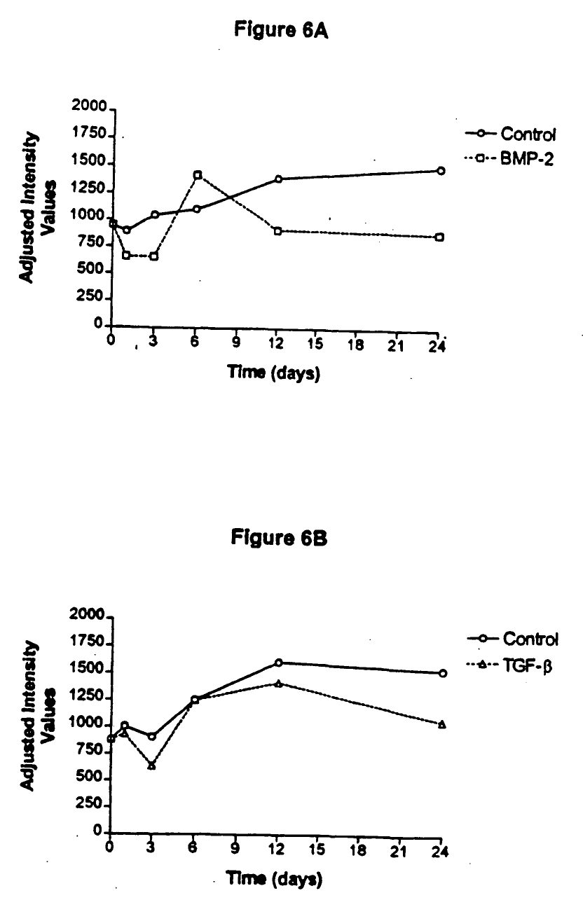

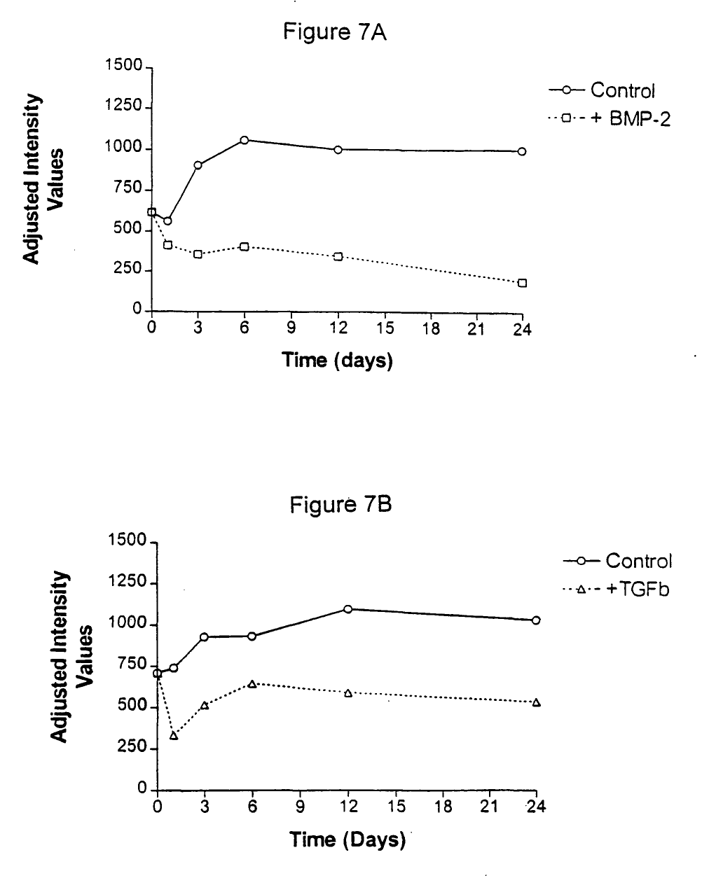

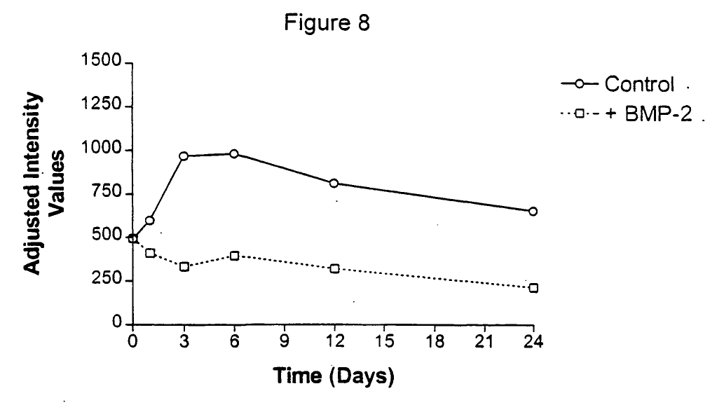

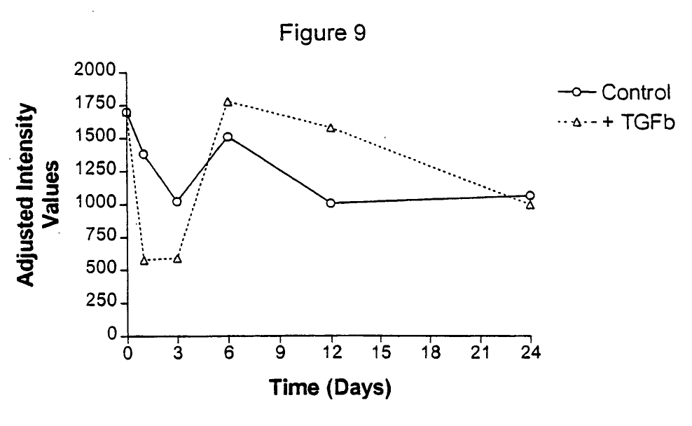

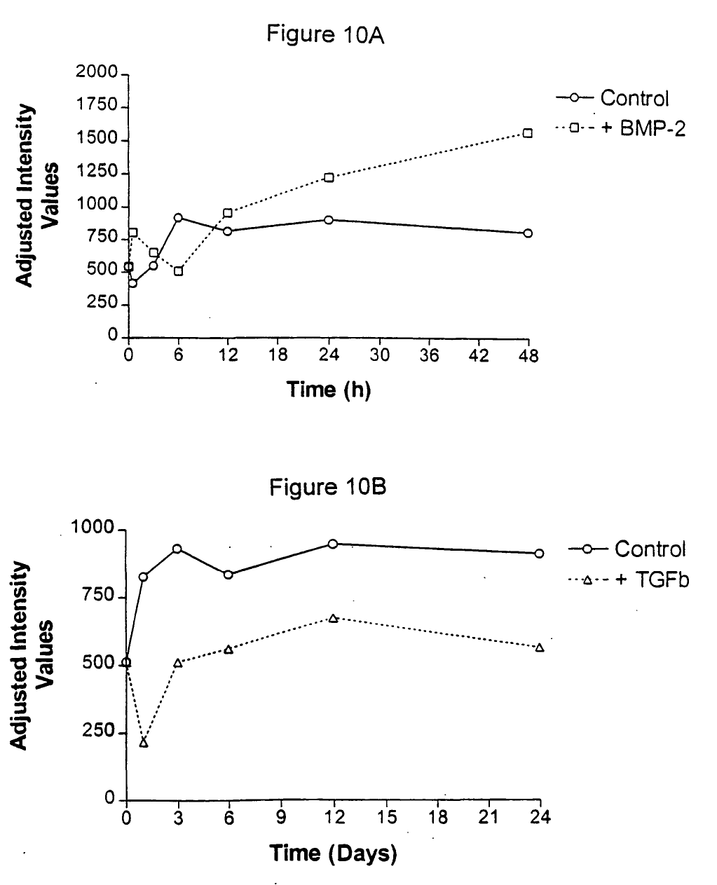

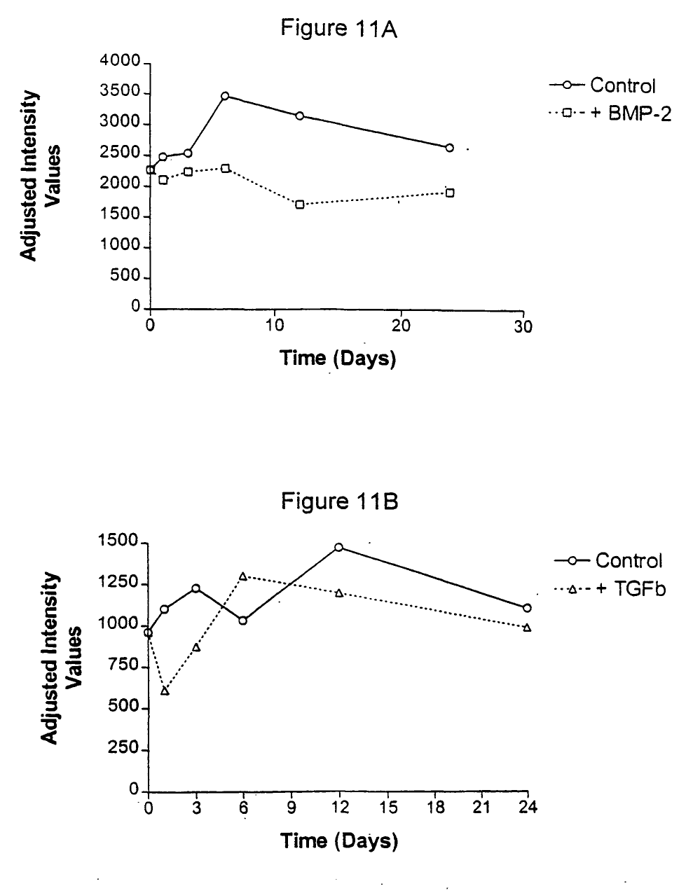

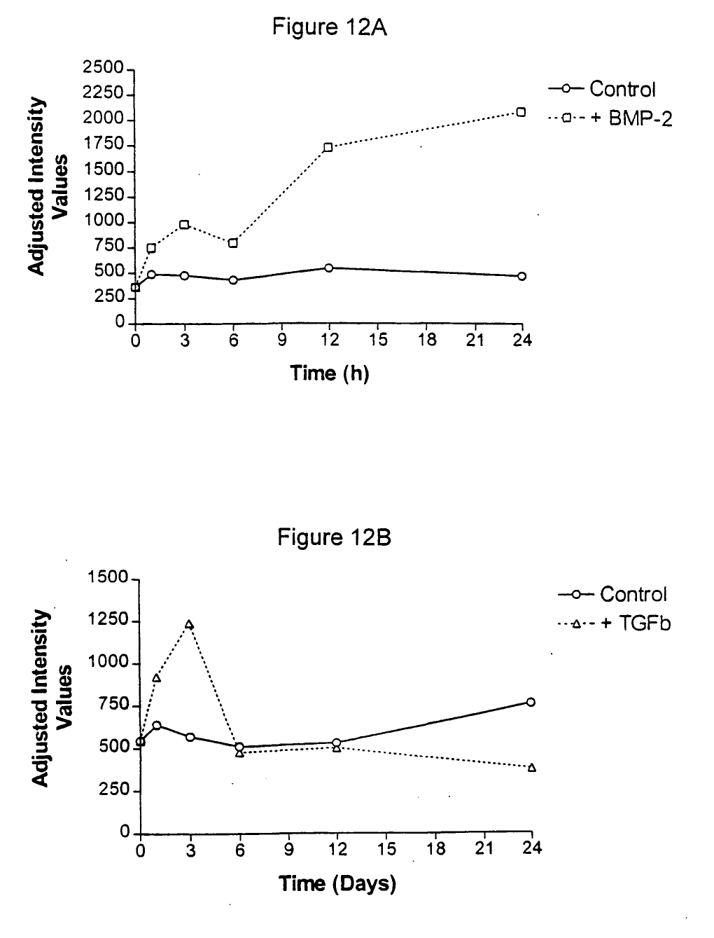

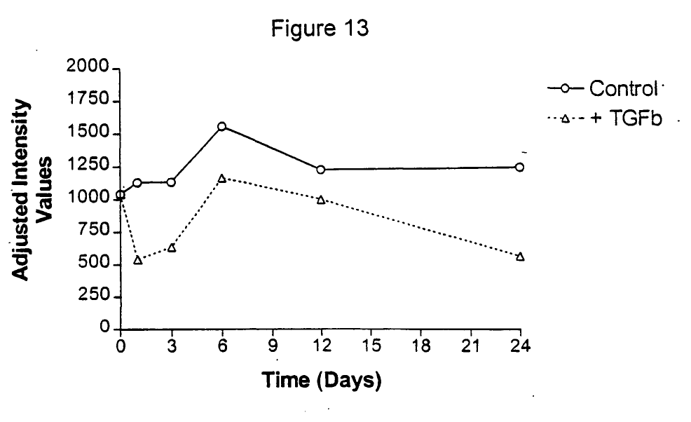

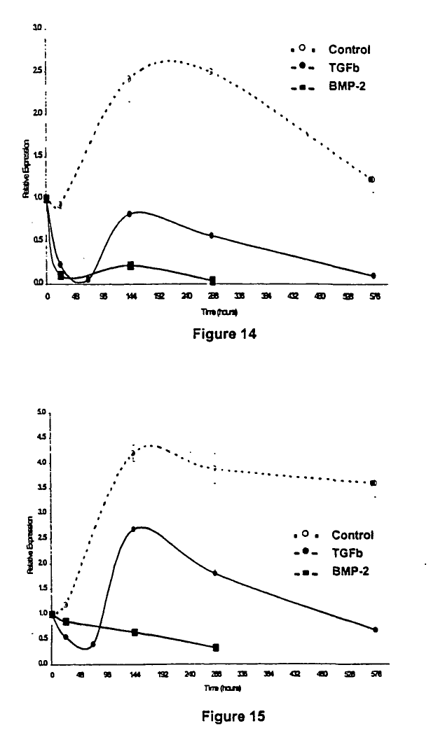

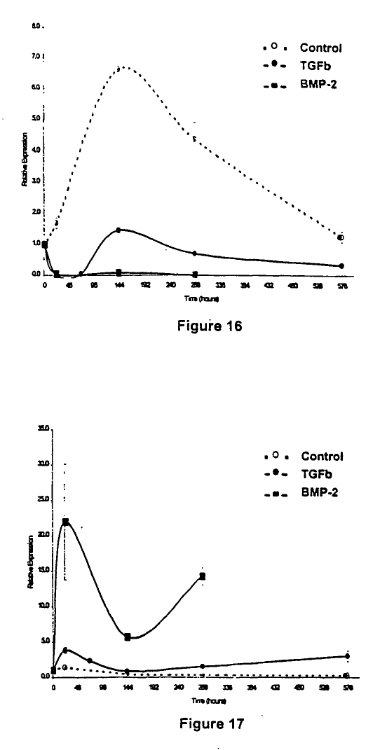

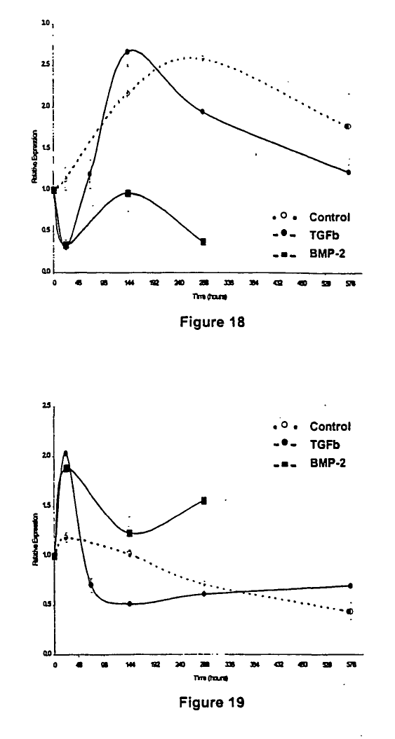

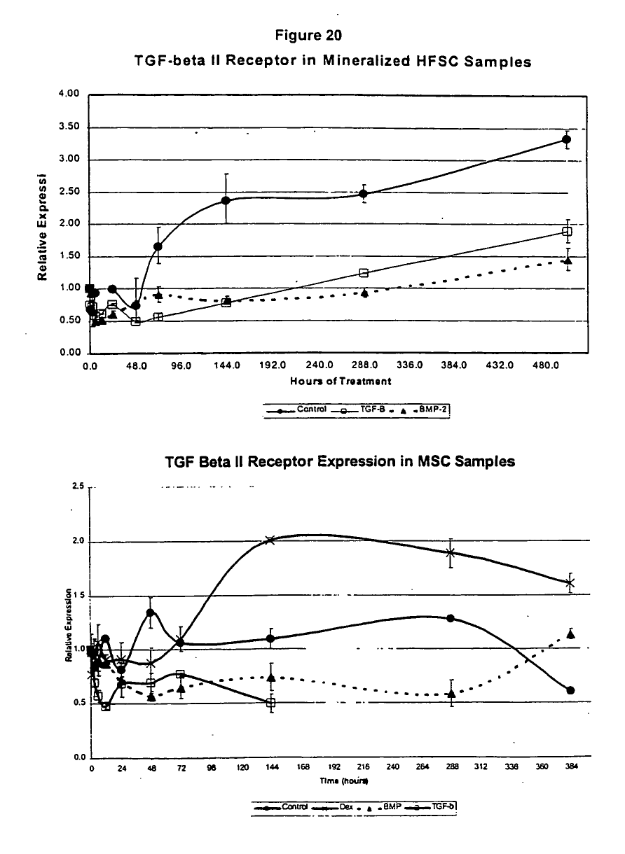

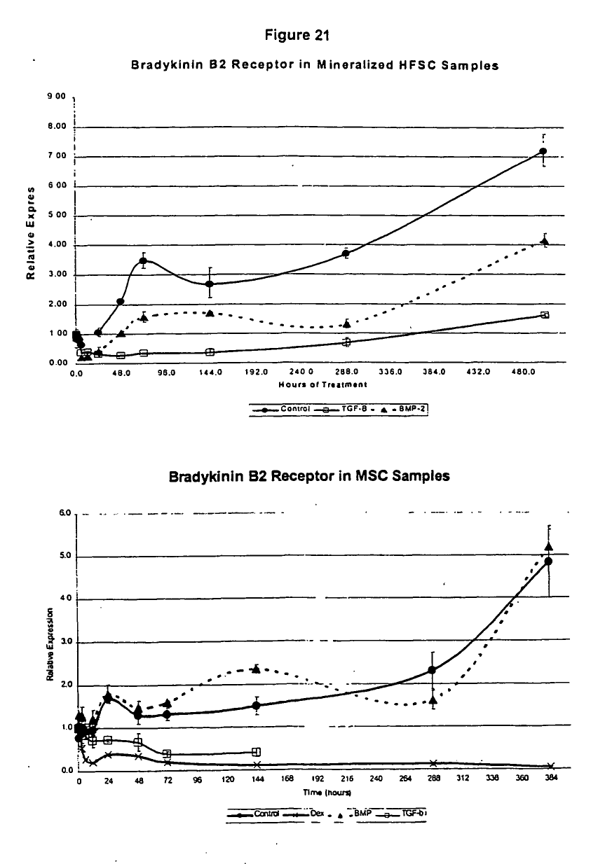

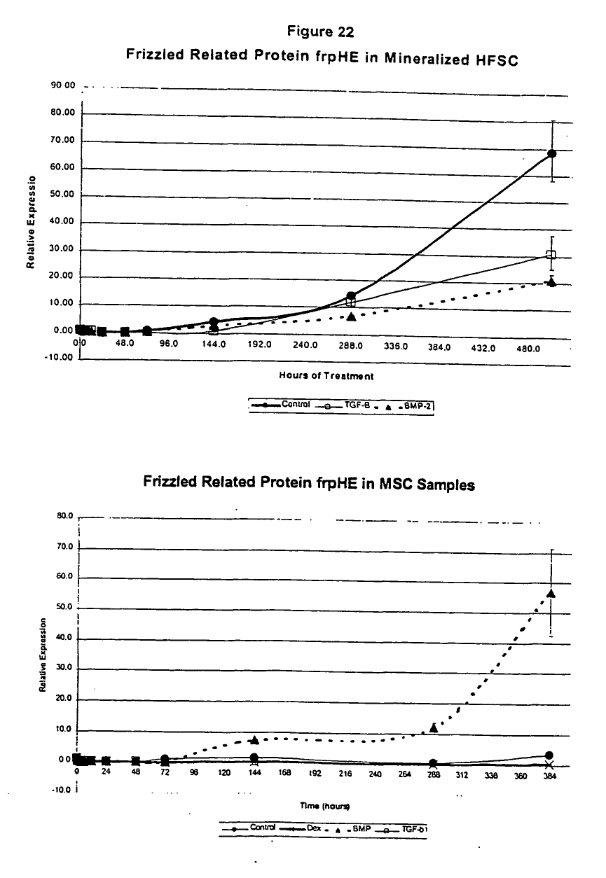

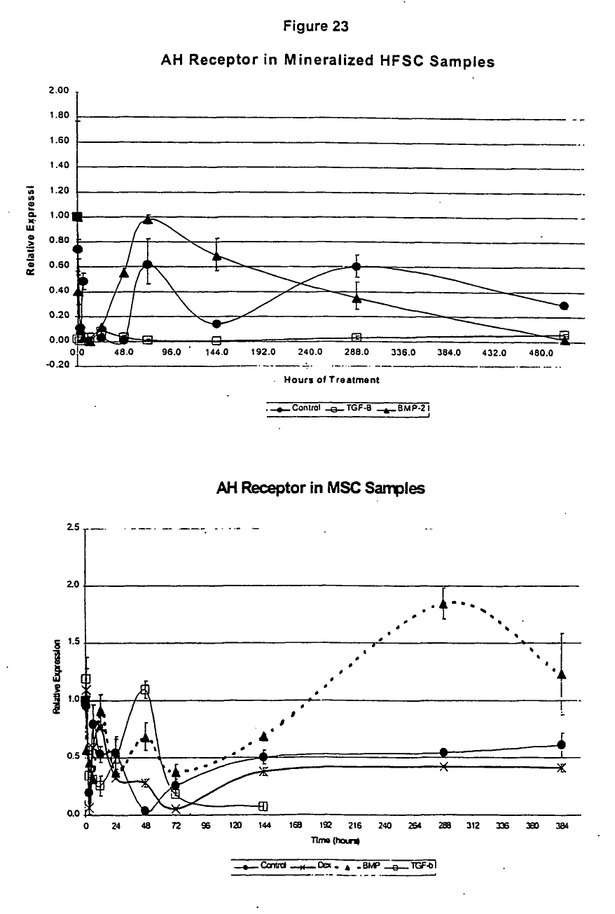

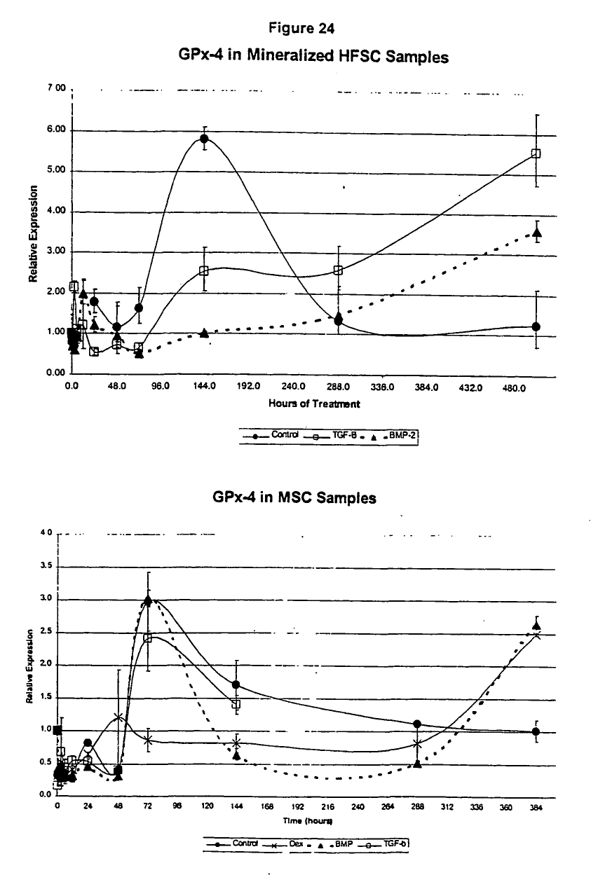

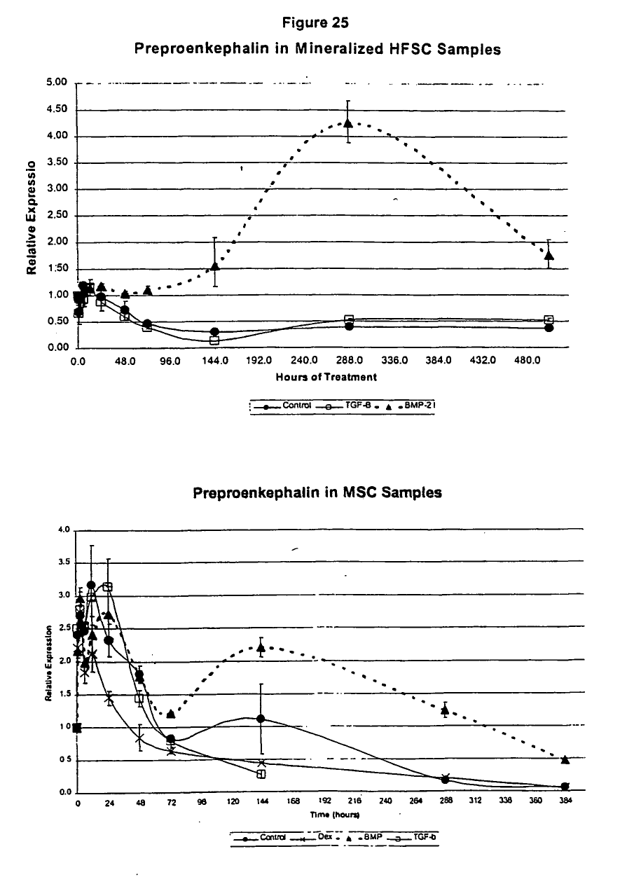

FIGS. 1-25 present a graphic depiction of the expression level of some genes whose expression pattern was found to be dependent upon the activation state of the precursor stem cells. These figures represent the data obtained from READS gel analysis of the mRNA expression data from Human Fetal Stromal Cells. READS analysis (as described above) was performed on the total RNA samples isolated from HFSCs that were treated with either TGFb (1 ng/ml of culture media) or BMP-2 (300 ng/ml of culture media) for up to twenty-four days. Time points were selected at one, three, six, twelve and twenty-four days post initial treatment. In a few cases, time points were selected at thirty minutes, three, six, twelve, twenty-four and forty-eight hours post initial treatment. Control cells received media only with no added osteogenic agent. Subsequent to READS gel analysis, the images of each gel were converted into electronic format and the intensities of each band of interest were calculated relative to the background autoradiographic intensity of each gel image. The corrected values are termed adjusted intensity values, which were plotted on the y-axis versus the time course of the experiment.

### Example 2

Gene Chip Expression Analysis

Precursor stem cells (for example, HFSCs or human mesenchymal stem cells) which may be treated with a differentiation inducing agent and/or osteoblasts may be obtained using any means known to those skilled in the art. For example, human mesenchymal stem cells (HMSCs) are isolated from human bone marrow and are capable of differentiating into bone, cartilage, fat and other connective tissues. HMSCs exposed to osteogenic stimulus undergo osteogenic differentiation by showing an increase in alkaline phosphatase (APase) enzyme activity and deposition of mineralized hydroxtapatite extracellular matrix (Jaiswal et al., (1997) J. Cell. Biochem 64:295-312). HMSCs obtained from Clonetics were expanded to passage four and cultured in a basal medium (DMEM-LG containing 10% FBS and 1% antibiotic/antimycotic) at 37° C. in a humidified atmosphere containing 95% air and 5% CO₂. Cultures were treated with BMP-2 (100 ng/ml) and TGFb1 (1 ng/ml) to extract RNA at twenty minutes, three, six, twelve, twenty-four, and forty-eight hours and, three, six, twelve and sixteen days.

Microarray sample preparation may be conducted following the protocols set forth in the Affymetrix GeneChip Expression Analysis Manual. For example, samples comprising cells of interest or tissue comprising such cells may be frozen. Frozen samples may be ground to a powder, for example, using a Spex Certiprep 6800 Freezer Mill. Total RNA may be extracted using conventional techniques such as with Trizol (GibcoBRL) utilizing the manufacturer's protocol. The total RNA yield for each sample will likely be in the range of 200-500 μg per 300 mg sample weight. mRNA may be isolated using the Oligotex mRNA Midi kit (Qiagen) followed by ethanol precipitation. Double stranded cDNA can be generated using conventional techniques such as those described above of by using the SuperScript Choice system (GibcoBRL). First strand cDNA synthesis may be primed with a T7-(dT24) oligonucleotide. The cDNA may be purified, i.e., may be phenol-chloroform extracted and ethanol precipitated. The cDNA may be re-suspended at final concentration of about 1 μg/ml. From 2 μg of cDNA, cRNA may be synthesized using Ambion's T7 MegaScript® in vitro Transcription Kit.

In preferred embodiments, the cRNA may be detectably labeled. The cRNA may be directly labeled by incorporating one or more detectable moieties into the cRNA molecule. In other embodiments, the cRNA may incorporate a moiety to which a detectably labeled reagent may bind. For example, the cRNA may incorporated a biotin or digoxigenin moiety and may be detected using a detectably labeled avid/streptavidin or anti-digoxigenin antibody. To incorporate a moiety to which a detectably labeled reagent may bind, nucleoside triphosphates containing the binding moiety may be added to the transcription reaction. For example, nucleotides Bio-11-CTP and Bio-16-UTP (Enzo Diagnostics) may be added to the reaction. The transcription reaction may be allowed to proceed an appropriate length of time in order to generate the desired amount of cRNA. Suitable conditions might be a 37° C. incubation for six hours. Typically, impurities can be removed from the cRNA using conventional techniques such as, for example, using the RNeasy® Mini kit protocol (Qiagen). cRNA can be fragmented by heating in a suitable buffer. One example of a suitable buffer would be of 200 mM Tris-acetate (pH 8.1), 500 mM KOAc and 150 mM MgOAc. The cRNA maybe heated at 94° C. for about thirty minutes.

The fragmented cRNA can be assayed using a gene chip. In some embodiments, the assay may be conducted using the Affymetrix protocol. For example, 55 μg of fragmented cRNA may be hybridized on the Affymetrix Human 42K array set for twenty-four hours at 60 rpm in a 45° C. hybridization oven. The chips may be washed and stained with a suitable reagent. When biotin is incorporated into the cRNA, one suitable reagent might be Streptavidin Phycoerythrin (SAPE) (Molecular Probes). To amplify staining, SAPE solution may be added twice with an anti-streptavidin biotinylated antibody (Vector Laboratories) staining step in between. Hybridization to the probe arrays may be detected using any technique known to those skilled in the art, for example, by fluorometric scanning using a Hewlett Packard Gene Array Scanner. Data may be analyzed using Affymetrix GeneChip® version 3.0 and Expression Data Mining Tool (EDMT) software (version 1.0). When the Affymetrix GeneChip 42K human gene chip is used to assay expression levels, the EDMT may be set to the following criteria: (1) For each gene, Affymetrix GeneChip average difference values may be determined by standard Affymetrix EDMT software algorithms, which also made “Absent” (=not detected), “Present” (=detected) or “marginal” (=not clearly Absent or Present) calls for each GeneChip element; (2) all negative values (=Absent) can be raised to a floor of +20 (positive 20) so that fold change calculations may be made where values were not already greater than or equal to +20; (3) median levels of expression may be compared between the differentiating and non-differentiating cells to obtain greater than or equal three-fold up/down values; (4) The median value for the higher expressing group may be greater or equal to 200 average difference units in order to be considered for statistical significance; (5) Genes passing the first four criteria will be analyzed for statistical significance using a two-tailed T test and deemed statistically significant if p<0.05.

The expression levels of one or more of the genes identified as involved in the differentiation of precursor stem cells may be assayed as described above. To serve as a positive control, the expression level of a gene that does not change during differentiation may be assayed.

### Example 3

Quantitative RT-PCR Verification of Expression Levels

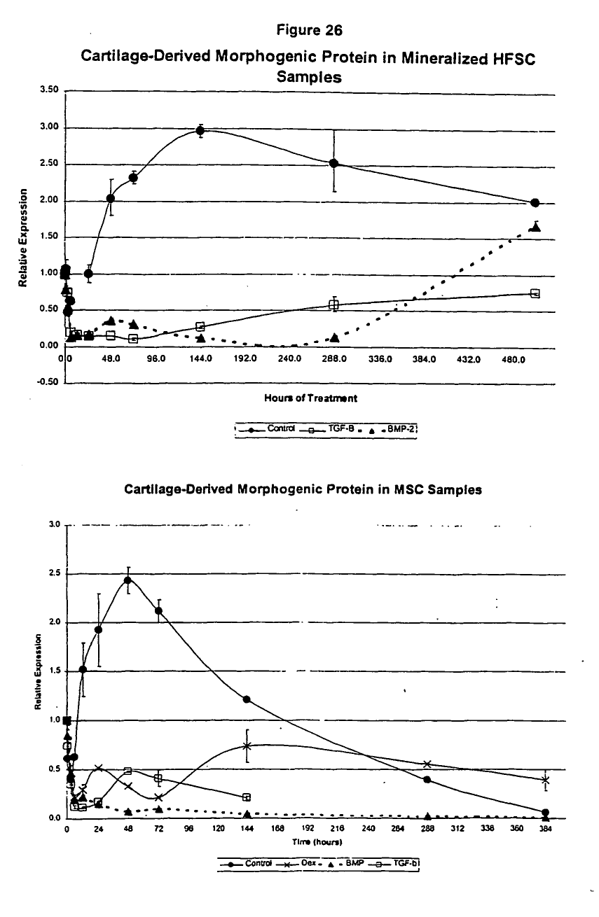

FIGS. 15-26 show quantitative RT-PCR profiles from some of the selected targets described in Table 1. Human fetal stromal cells (HFSCs) and Human Mesenchymal stem cells (HMSCs) were used for this study. Briefly, PCR primers were designed using the DNA sequences provided by sequence analysis of the READS fragments. TaqMan probes were also designed using the READS fragment sequence information. Experimental conditions were as follows: HFSCs were cultures in vitro and were left untreated for up to twenty days, or were treated with the osteogenic agents TGFb (1 ng/ml culture media) or BMP-2 (300 ng/ml of culture media) for the same period. HMSCs were also cultured in vitro and were left untreated for sixteen days, or were treated with TGFb (1 ng/ml culture media), BMP-2 (300 ng/ml culture media) or dexamethasone (100 nM) for the same time period. Cells in each treatment group were harvested at zero, three, six and twelve hours, one, three, six, twelve and twenty days in the case of HFSCs. For the HMSC experiments, cells were harvested at zero, three, six and twelve hours, one, three, six, twelve and sixteen days post treatment. Total RNA was isolated from the cells using Trizol and the RNA was quantitated using a spectrophotometer set at A₂₆₀. Total RNA (10 ng) was assayed in duplicate suing the TaqMan® assay (Perkin-Elmer) in biplex format where each target gene in each RNA sample was assayed versus a reference mRNA which was shown previously to be constitutively expressed and not regulated by any of the osteogenic treatments. The threshold cycle (C_{T}) values of the target and reference gene were analyzed and the delta CT values were calculated for each RNA sample. Fold change (expressed as relative expression) was plotted versus the time course of the experiment. Expression was relative to the delta CT value (Target CT minus Reference CT) for t=0 which was set to a value of 1.0.

### Example 4

Activity Assays

The present invention has identified numerous genes and gene families differentially regulated during the differentiation of precursor stem cells into osteoblasts. The activity levels of proteins encoded by these genes or members of gene families may be assayed using any technique known to those skilled in the art. When the encoded protein is an enzyme, it may be desirable to assay the enzymatic activity of the protein. This may accomplished, for example, by contacting a sample with a substrate for the enzyme and assaying for the conversion of substrate to product. For example, a labeled substrate may be provided which is converted into a labeled product which may be subsequently quantified. Labels may be of any type conventionally used by those skilled in the art for this purpose. In some preferred embodiments, the label may be a chromophoric group, a fluorescent group, a radioactive group or other detectable group.

In some instances it may be preferable to detect the activity using an immunological technique such as Western blotting, ELISA, radio-immunoprecipitation (RIP) and the like. Thus, the term activity is seen to include the physical presence of the protein of interest. This may be useful in cases where the protein lacks a readily assayable enzymatic activity or where, for other reasons, assaying an enzymatic activity is not desirable.

An agent which may be an activator or inhibitor of a particular biological target may be assayed. The assays may be cell-free assays to measure the biological activity of protein target after disruption of the cell in which the target is expressed. The assays may be cell based assays to determine the activity of the target protein by measuring a biological response of a cell in which the target protein is located.

Cell-free assays may optionally include one or more purification steps. Such purification steps include, but are not limited to, centrifugation steps, precipitation steps and chromatographic steps. After disruption of the cell expressing the protein target of interest, the target may be purified to a desired purity before the assay is conducted. When the assay is specific for the activity in question, it may be desirable to use the disrupted cells with no purification step. In other instances, it may be desirable to purify the desired activity from one or more contaminants prior to assaying. In a preferred cell-free system, enzyme activity or receptor binding may be measured using europhium-chelated antibody specific for target enzyme or europhium-derivitized ligand that binds to receptor (see, for example, Mathis, (1993) Clin. Chem. 39:1953-1959; Gaarde et al., (1997) J. Biomol. Screen. 2:213-223). In some embodiments, fluorescence polarization/correlation spectroscopy can also be used to measure enzymatic or binding reaction by using fluorescenylated peptide substrate or target (Seethala et al., (1997) Anal. Biochem, 253:210-218, 1997, Lynch et al., (1997) Anal Biochem 247:77-82).

Cell-based assays using reporter genes may be used for the screening of compounds. Activation of a cell surface receptor or a ligand-gated ion channel can induce a change in the transcription pattern of a number of genes. The ligand-induced alteration in transcription can be measured using gene fusion, in which a promoter element responsive to activation is fused to the coding region for an enzyme or protein whose level can easily be measured (Martin et al., (1996) Biotechniques 21:520-524). Other assays, which detect immediate early response to gene activation are—elevation of second messengers (cAMP, Ca²⁺), phosphorylation of an intermediate signaling protein or subcellular translocation of a signaling molecule (Stable et al., (1997) Anal. Biochem. 252:115-126; Miyawaki et al., (1997) Nature 388:882-887; Lenormand et al., (1993) J. Cell. Biol. 122:1079-1088). Design and execution of such assays are routine in the art.

### Example 5

Drug Screening Assays

Candidate agents and compounds will be screened for their ability to modulate the expression levels and/or activities of one or more of the genes identified as being involved in the differentiation of precursor stem cells into osteoblasts by any technique known to those skilled in the art including those assays described above. In some preferred embodiments, the assay of gene expression level may be conducted using real time PCR. Real time PCR detection may be accomplished by the use of the ABI PRISM 7700 Sequence Detection System. This system measures the fluorescence intensity of the sample each cycle and is able to detect the presence of specific amplicons within the PCR reaction. Each sample is assayed for the level of one or more of the genes identified as being involved in the differentiation of precursor cells into osteoblasts including, but not limited to, those genes and members of gene families identified in Table 1.

The expression level of a control gene, for example GAPDH, may be used to normalize the expression levels. Suitable primers for the candidate genes may be selected using techniques well known to those skilled in the art. These primers may be used in conjunction with SYBR green (Molecular Probes), a nonspecific double stranded DNA dye, to measure the expression level mRNA corresponding to the genes, which will typically be normalized to the GAPDH level in each sample.

Normalized expression levels from cells exposed to the agent are then compared to the normalized expression levels in control cells. Agents that modulate the expression of one or more the genes may be further tested as drug candidates in appropriate in vitro and/or in vivo models.

Although the present invention has been described in detail with reference to examples above, it is understood that various modifications can be made without departing from the spirit of the invention. Accordingly, the invention is limited only by the following claims. All cited patents, applications and publications referred to in this application are herein incorporated by reference in their entirety.

| SEQ. | | | | | Expression |
|---|---|---|---|---|---|
| ID | Class | GB Number | GB Name | Identity | Profile |
| 1 | MatchGene | X53390 | Human upstream binding factor | 238/241 98 | Upregulated |
| 2 | | X53461 | Human upstream binding factor | 238/241 98 | Upregulated |
| 3 | | AC004596 | Homo sapiens chromosome 17, clone | 234/236 99 | Upregulated |
| | | | hCIT.175_E_5 | | |
| 4 | MatchGene | AF155135 | Homo sapiens novel retinal pigment | 479/484 98 | Upregulated |
| | | | epithelial cell protein | | |
| 5 | | AB037755 | Homo sapiens | 479/484 98 | Upregulated |
| | | | KIAA1334 protein | | |
| 6 | MatchGene | AF153606 | Homo sapiens angiopoietin- | 388/416 93 | Upregulated |
| | | | related protein | | |
| 7 | | AF169312 | Homo sapiens hepatic angiopoietin- | 136/137 99 | Upregulated |
| | | | related protein (ANGPTL2) | | |
| 8 | MatchGene | AL078459 | Human clone RP4-621F18chr.1p11.4- | 435/438 99 | Upregulated |
| | | | 21.3.3 ng,ng dimethylarginine | | |
| | | | dimethylaminohydrolase | | |
| 9 | | AB001915 | Homo sapiens cDNA for NG,NG- | 73/73 100 | Upregulated |
| | | | dimethylarginine dimethylaminohydrolase | | |
| 10 | MatchGene | X17620 | Human mRNA for Nm23 protein | 500/504 99 | Upregulated |
| 11 | | X75598 | Homo sapiens nm23H1 gene | 309/310 99 | Upregulated |
| 12 | | AL360191 | Homo sapiens full length insert | 503/508 99 | Upregulated |
| | | | cDNA clone EUROIMAGE 781354 | | |
| 13 | | AL360166 | Homo sapiens mRNA full length | 502/508 98 | Upregulated |
| | | | insert cDNA clone EUROIMAGE | | |
| | | | 300041 | | |
| 14 | MatchGene | AF038633 | Homo sapiens Mpv17 gene and | 201/235 85 | Downregulated |
| | | | urocortin gene | | |
| 15 | | X76538 | Homo sapiens Mpv17 | 197/232 84 | Downregulated |
| 16 | MatchGene | X07577 | Human mRNA for C1 inhibitor | 251/252 99 | Downregulated |
| 17 | | X07433 | Homo sapiens for C1 inhibitor | 251/252 99 | Downregulated |
| | | | exon 8 | | |
| 18 | | M13656 | Human plasma protease (C1) | 250/252 99 | Downregulated |
| | | | inhibitor | | |
| 19 | | X54486 | Human gene for C1 inhibitor | 253/254 99 | Downregulaled |
| 20 | | M13690 | Human plasma protease (C1) | 246/253 97 | Downregulated |
| | | | inhibitor | | |
| 21 | MatchGene | X66924 | Homo sapiens heir-1 mRNA for | 486/540 90 | Upregulated |
| | | | helix-loop-helix protein | | |
| 22 | | X69111 | Homo sapiens HLH 1R21 mRNA for | 464/514 90 | Upregulaled |
| | | | helix-loop-helix protein | | |
| 23 | | X73428 | Homo sapiens Id3 gene for HLH | 459/525 87 | Upregulated |
| | | | type transcription factor | | |
| 24 | | AL021154 | Homo sapiens PAC 150O5 chr. | 460/525 87 | Upregulated |
| | | | 1p36.13-36.22 Contains E2F2 | | |
| | | | gene and ID3 gene | | |
| 25 | MatchGene | S63168 | CCAAT/enhancer-binding protein | 278/278 100 | Downregulated |
| | | | delta = transcription factor | | |
| | | | CRP3 homolog | | |
| 26 | | M83667 | Human NF-IL6-beta protein | 273/279 97 | Downregulated |
| 27 | MatchGene | AF109127 | Homo sapiens stromal cell-derived | 252/269 93 | Upregulated |
| | | | receptor-1 alpha | | |
| 28 | | AF109126 | Homo sapiens stromal cell- | 252/269 93 | Upregulated |
| | | | derived receptor-1 alpha | | |
| 29 | | AF035287 | Homo sapiens clone 23742 | 253/269 94 | Upregulated |
| 30 | MatchGene | M90357 | Human basic transcription | 99/101 98 | Downregulated |
| | | | factor 3a (BTF3a) | | |
| 31 | | X74070 | Homo sapiens mRNA for | 87/89 97 | Downregulated |
| | | | transcription factor BTF3 | | |
| 32 | | AC000403 | Genomic sequence for Human 13 | 96/104 92 | Downregulated |
| 33 | | AL121766 | Human chromosome 14 BAC R- | 81/91 89 | Downregulated |
| | | | 412H8 library RPCI-11 | | |
| 34 | MatchGene | Y11307 | Homo sapiens CYR61 mRNA | 273/274 99 | Upregulated |
| 35 | | Z98053 | Homo sapiens mRNA for hCYR61 | 269/270 99 | Upregulated |
| | | | protein | | |

TABLE 1

| | | | | | |
|---|---|---|---|---|---|
| 36 | | Y12084 | Homo sapiens mRNA for gig 1 protein | 269/270 99 | Upregulated |
| 37 | | U62015 | Homo sapiens Cyr61 mRNA | 269/270 99 | Upregulated |
| 38 | | AF031385 | Homo sapiens Cyr61 protein (CYR61) mRNA | 269/270 99 | Upregulated |
| 39 | MatchGene | AF015257 | Homo sapiens flow-induced endothelial G | 376/378 99 | Upregulated |
| | | | protein-coupled receptor (FEG-1) | | |
| 40 | | U63917 | Human GPCR-Br | 376/380 98 | Upregulated |
| 41 | MatchGene | AF177394 | Homo sapiens dickkopf-1 (DKK-1) | 243/243 100 | Upregulated |
| 42 | | AF127563 | Homo spiens Sk/Dkk-1 protein precursor | 243/243 100 | Upregulated |
| 43 | MatchGene | U17077 | Human BENE | 140/143 97 | Upregulated |
| 44 | | U17079 | Homo sapiens clone P607 4-4 non-V kappa | 131/14292 | Upregulated |
| | | | IgK | | |
| 45 | MatchGene | D83402 | Homo sapiens gene for prostacyclin synthase | 362/373 97 | Downregulated |
| 46 | | AL118525 | Homo sapiens sequence from clone RP11-298O6 | 364/373 97 | Downregulated |
| | | | on chromosome 20 | | |
| 47 | | AC006957 | Homo sapiens chromosome 5q, BAC clone 104n10 | 306/322 95 | Downregulated |
| | | | (LBNL H295) | | |
| 48 | MatchGene | L43821 | Homo sapiens enhancer of filimentation (HEF1) | 372/399 93 | Upregulated |
| 49 | | U64317 | Human Crk-associated substrate related protein | 177/179 98 | Upregulated |
| | | | Cas-L | | |
| 50 | | AL136139 | Human clone RP4-761I2 on chromosome 6.3′ | 372/396 93 | Upregulated |
| | | | HEF1 | | |
| 51 | MatchGene | AJ245902 | Homo sapiens partial mRNA for Claudin-11 | 400/401 99 | Downregulated |
| | | | protein (CLDN11 gene) | | |
| 52 | | AF085871 | Homo sapiens full length insert cDNA clone | 404/405 99 | Downregulated |
| | | | Y011A01 | | |
| 53 | | AC008041 | Homo sapiens 3q26.2-27 BAC RPCI11-469J4 | 404/405 99 | Downregulated |
| 54 | MatchGene | AF073515 | Homo sapiens cytokine type-1 receptor | 128/128 100 | Upregulated |
| | | | precursor | | |
| 55 | | AF178684 | Homo sapiens class I cytokine receptor | 126/126 100 | Upregulated |
| | | | (zcytor5) | | |
| 56 | | AF059293 | Homo sapiens cytokine-like factor-1 | 125/125 100 | Upregulated |
| | | | precursor | | |
| 57 | | AC003112 | Human DNA chromosome specific cosmid | 125/125 100 | Upregulated |
| | | | R30292 | | |
| 58 | MatchGene | AF182032 | Homo sapiens protein kinase inhibitor | 96/98 97 | Downregulated |
| | | | gamma | | |
| 59 | | AB019517 | Homo sapiens PKIG | 95/98 96 | Downregulated |
| 60 | | M13792 | Human adenosine deaminase gene | 93/94 98 | Downregulated |
| 61 | | Z97053 | Human DMA sequence clone RP1-179M20 on | 93/94 98 | Downregulated |
| | | | chromosome 20 | | |
| 62 | MatchGene | AB018356 | Homo sapiens mRNA for GM3 synthase | 415/416 99 | Downregulated |
| 63 | | AF119415 | Homo sapiens GM3 synthase | 417/420 99 | Downregulated |
| 64 | | AF119418 | Homo sapiens nonfunctional GM3 synthase | 417/420 99 | Downregulated |
| 65 | | AF119417 | Homo sapiens nonfunctional GM3 synthase | 417/420 99 | Downregulated |
| 66 | | AF161396 | Homo sapiens HSPC278 | 419/420 99 | Downregulated |
| 67 | MatchGene | AF200348 | Homo sapiens melanoma-associated antigen | 131/132 99 | Upregulated |
| | | | MG50 | | |
| 68 | | AL109748 | Homo sapiens chromosome 21 PAC RPCIP704 | 55/74 74 | Upregulated |
| | | | M13256Q2 | | |
| 69 | MatchGene | M22960 | Human protective protein | 211/212 99 | Downregulated |
| 70 | | AF086236 | Homo sapiens full length insert cDNA | 215/217 99 | Downregulated |
| | | | clone ZD20E11 | | |
| 71 | | AL008726 | Human sequence from clone RP3-337O18 | 213/215 99 | Downregulated |
| | | | on chromosome 20q12-13.1 | | |
| 72 | | AF052157 | Homo sapines clone 24801 mRNA | 62/63 98 | Downregulated |
| 73 | | L26232 | Human phospholipid transfer protein | 60/61 98 | Downregulated |
| 74 | MatchGene | U03877 | Human extracellular protein (S1-5) | 344/348 98 | Downregulated |
| 75 | MatchGene | AB035480 | Homo sapiens mRNA for type 1 | 482/489 98 | Upregulated |
| | | | transmembrane protein | | |
| 76 | | AC004643 | Homo sapiens chromosome 16, cosmid | 485/488 99 | Upregulated |
| | | | clone 363E3 (LANL) | | |
| 77 | | AF191148 | Homo sapiens type 1 transmembrane | 484/489 98 | Upregulated |
| | | | protein Fn14 | | |
| 78 | MatchGene | D84342 | Homo sapines for SM22 alpha | 429/439 97 | Upregulated |
| 79 | | M95787 | Human 22 kDa smooth muscle protein | 609/621 98 | Upregulated |
| 80 | | D17409 | Homo sapiens mRNA for SM22 alpha | 612/621 98 | Upregulated |
| 81 | MatchGene | AF166125 | Homo sapiens selenoprotein N | 90/96 93 | Downregulated |
| 82 | | AL110205 | Homo sapiens mRNA; cDNA DKFZp586L1722 | 91/96 94 | Downregulated |
| 83 | | AL020996 | Human DNA sequence clone RP1-317E23 | 89/94 94 | Downregulated |
| | | | on chromosome 1p36.13. | | |
| 84 | | AC010485 | Homo spiens chromosome 19 clone CTD- | 58/88 65 | Downregulated |
| | | | 2329C7 | | |
| 85 | MatchGene | L11066 | Human mRNA sequence (peptide-binding | 155/155 100 | Upregulated |
| | | | protein 74) | | |
| 86 | | AL035091 | Human DNA sequence form clone 8L2 on | 146/154 94 | Upregulated |
| | | | chromosome 1q32.2-q32.3 contains CD34 | | |
| 87 | MatchGene | X51521 | Human mRNA for ezrin | 143/144 99 | Upregulated |
| 88 | | J05021 | Human cytovillin 2 (VIL2) | 73/74 | Upregulated |
| 89 | | AL162086 | Homo sapiens cDNA DKFZp762H157 | 151/155 97 | Upregulated |
| 90 | MatchGene | U89942 | Human lysyl oxidase-related protein | 362/365 99 | Upregulated |
| | | | (WS9-14) | | |
| 91 | | AF117949 | Homo sapiens lysyl oxidase-like | 364/370 98 | Upregulated |
| | | | protein 2 (LOXL2) | | |
| 92 | MatchGene | X76534 | Homo sapiens NMB mRNA | 401/401 100 | Downregulated |
| 93 | | AC005082 | Homo sapiens clone RG271G13 | 401/401 100 | Downregulated |
| 94 | MatchGene | U07802 | Human Tis11d gene | 215/219 98 | Downregulaled |
| 95 | MatchGene | X14958 | Human high mobility group protein | 142/143 99 | Downregulated |
| 96 | | X14957 | Human high mobility group protein | 142/143 99 | Downregulated |
| 97 | | M23615 | Human HMG-Y protein isoform | 139/139 100 | Downregulated |
| 98 | | M23617 | Human HMG-Y protein isoform | 139/139 100 | Downregulated |
| 99 | | M23618 | Human HMG-Y protein isoform | 139/139 100 | Downregulated |
| 100 | MatchGene | M64347 | Human novel growth factor receptor | 249/249 100 | Upregulated |
| 101 | MatchGene | M76979 | Homo sapiens pigment epithelium- | 647/663 97 | Downregulated |
| | | | differentiation factor | | |
| 102 | | M90439 | Human molecular marker (EPC-1) | 646/663 97 | Downregulated |
| | | | gene | | |
| 103 | | U29953 | Human pigment epithelium derived | 354/365 96 | Downregulated |
| | | | factor | | |
| 104 | MatchGene | AL133105 | Homo sapiens; cDNA DKFZp434F2322 | 266/272 97 | Downregulated |
| 105 | | AC005799 | Homo sapiens chromosome 17, clone | 264/271 97 | Downregulated |
| | | | hRPK.62 F 10 | | |
| 106 | MatchGene | AL049389 | Homo sapiens cDNA DKFZp586O0118 | 333/335 99 | Downregulated |
| 107 | | AL109718 | Homo sapiens clone EUROIMAGE | 330/332 99 | Downregulated |
| | | | 668307 | | |
| 108 | | AK000465 | Homo sapiens cDNA FLJ20458 fis, | 330/332 99 | Downregulated |
| | | | clone KAT05904 | | |
| 109 | | AL359061 | Homo sapiens clone-EUROIMAGE 2155535 | 329/331 99 | Downregulated |
| 110 | | AB033025 | Homo sapiens mRNA for KIAA1199 protein | 329/331 99 | Downregulated |
| 111 | MatchGene | AL080214 | Homo sapiens mRNA; cDNA DKFZp586l2223 | 259/261 99 | Downregulated |
| 112 | | AK000710 | Homo sapiens cDNA FLJ20703 fis, clone | 262/266 98 | Downregulated |
| | | | KAIA1965 | | |
| 113 | | AF124432 | Homo sapiens HOM-TES-103 tumor antigen | 258/261 98 | Downregulated |
| 114 | | AC006064 | Homo sapiens 12p13.3 PAC RPCI5-940J5 | 259/261 99 | Downregulated |
| 115 | MatchGene | AF052094 | Homo sapiens clone 23698 | 280/280 100 | Upregulated |
| 116 | | AF119117 | Homo sapiens dopamine transporter | 54/73 73 | Upregulated |
| 117 | MatchGene | AL050228 | Homo sapiens mRNA; cDNA DKFZp586O2223 | 343/345 99 | Upregulated |
| 118 | Related To | M15917 | Rat serine protease inhibitor 1 mRNA | 139/146 95 | Upregulated |
| 119 | | D00752 | Rat mRNA for contrapsin-like protease | 137/137 100 | Upregulated |
| | | | inhibotor related protein (Cpi-23) | | |
| 120 | | X16357 | Rat mRNA for SPI-1 serine protease | 139/147 94 | Upregulated |
| | | | Inhibitor | | |
| 121 | Related To | L35108 | Rattus norvegicus water channel protein | 183/260 70 | Downregulated |
| | | | (AQP3) gene | | |
| 122 | | D17695 | Rat mRNA for water channel aquaporin 3 | 180/255 70 | Downregulated |
| | | | (AQP3) | | |
| 123 | | AF104416 | Mus musculus aquaporin-3 mRNA | 117/172 68 | Downregulated |
| 124 | Related To | S78079 | Scleraxis + basic helix − loop − helix | 117/161 72 | Upregulated |
| | | | transcription factor [mice, embryos, | | |
| | | | mRNA] | | |
| 125 | Related To | D14313 | Chicken mRNA for deltaEF1 | 196/214 91 | Downregulated |
| 126 | | D76434 | Chicken gene for transcriptlonal repressor | 201/220 91 | Downregulated |
| | | | deltaEF1, exon 2-exon 9. | | |
| 127 | | L13856 | Mesocricetus auratus (hamster) DNA binding | 190/215 88 | Downregulated |
| | | | protein (BZP) mRNA | | |
| 128 | Related To | AF012923 | Mus musculus p53-inducible zinc finger | 227/326 69 | Downregulated |
| | | | protein (Wig-1) mRNA | | |
| 129 | MatchEST | N21464 | yx57d09.s1 Soares melanocyte 2NbHM | 470/504 93 | Downregulated |
| | | | Homo sapiens cDNA clone | | |
| 130 | MatchEST | AI081618 | ow77c02.s1 Soares fetal liver spleen 1NFLS | 231/236 97 | Upregulated |
| | | | S1 Homo sapiens cDNA clone | | |
| 131 | MatchEST | AI869864 | w m07c12.x1 NCI CGAP Ut4 Hs cDNA clone | 111/111 100 | Downregulated |
| | | | IMAGE: 2435254 3′ | | |
| 132 | MatchEST | AI769341 | w g36g07.x1 Soares NSF F8 9W OT PA P S1 | 186/186 100 | Upregulated |
| | | | Homo sapiens cDNA clone similar to gb: | | |
| | | | S65738 DESTRIN (Human) | | |
| 133 | MatchEST | AI343901 | qp12h11.x1 NCI CGAP KidS Homo sapiens | 342/371 92 | Upregulated |
| | | | cDNA clone | | |
| 134 | MatchEST | AA808622 | oe56c12.s1 NCI CGAP Lu5 Homo sapiens | 151/152 99 | Downregulated |
| | | | cDNA clone | | |
| 135 | MatchEST | AA564610 | nj02f05.s1 NCI CGAP Pr21 Homo sapiens | 128/130 98 | Downregulated |
| | | | cDNA clone | | |
| 136 | MatchEST | AA722810 | zg81h01.s1 Soares fetal heart NbHH19W | 307/307 100 | Upregulated |
| | | | Homo sapiens cDNA | | |
| 137 | MatchEST | AA417890 | zv05d12.s1 Soares NhHMPu S1 Homo sapiens | 247/248 99 | Downregulated |
| | | | cDNA clone | | |
| 138 | MatchGene | D50683 | Homo sapiens mRNA for TGF-betallR alpha | 343/348 98 | Downregulated |
| 139 | MatchGene | X86163 | H. sapiens mRNA for B2-bradykinin receptor | 204/209 97 | Downregulated |
| 140 | RelatedTo | AF026692 | Homo sapiens frizzled related protein | 66/103 64 | Downregulated |
| | | | frpHE mRNA | | |
| 141 | RelatedTo | E28892 | Member of FRZB family. FRAZZLED | 48/58 82 | Downregulated |
| 142 | MatchGene | L19872 | Human AH-receptor mRNA | 230/233 98 | Downregulated, Up- |
| 143 | MatchGene | X71973 | H. sapiens GPx-4 mRNA for phospholipid | 78/80 97 | Downregulated |
| | | | hydroperoxide glutathione peroxidase | | |
| 144 | MatchGene | AF060973 | HS selenium-dependent phospholipid | 72/73 98 | Downregulated |
| | | | hydroperoxide glutathione peroxidase | | |
| 145 | MatchGene | V00510 | Human gene for preproenkephalin | 582/602 96 | Upregulated |
| 146 | MatchGene | J00123 | Human enkeptialin gene | 582/602 97 | Upregulaled |
| 147 | MatchGene | K00469 | human enkephalin gene | 581/601 96 | Upregulated |
| 148 | MatchGene | U13660 | Human cartilage-derived morphogenetic | 601/627 96 | Downregulated |
| | | | protein 1 (CDMP-1) mRNA | | |
| 149 | MatchGene | X80915 | HS Gdf5 gene | 605/627 96 | Downregulated |

What is claimed is:

1. A method of screening for an agent that modulates the differentiation of precursor stem cells into osteoblasts, comprising:
(a) preparing a first gene or gene family expression profile of a cell population comprising precursor stem cells and/or assaying an activity of a protein encoded by at least one gene or a member of a gene family of Table 1 of a cell population comprising precursor stem cells;
(b) exposing the cell population to the agent;
(c) preparing second gene or gene family expression profile of the agent exposed cell population and/or assaying an activity of a protein encoded by at least one gene or a member of a gene family of Table 1 of the exposed cell population; and
(d) comparing the first and second expression profiles or first and second activities to an expression profile and/or an activity of an osteoblast cell population.

2. A method of claim 1, wherein the gene expression profiles comprise the expression levels for a set of genes that are differentially regulated in precursor stem cells compared to osteoblasts.

3. A method of claim 1, wherein the agent modulates the level of expression or activity for at least one gene in the precursor stem cell population to the expression level found in an osteoblast cell population.

4. A method of claim 1, wherein the gene expression profiles or activity level comprise the expression or activity levels in a cell of at least two genes or members of a gene family in Table 1.

5. A method of diagnosing a condition characterized by abnormal deposition of bone tissue, comprising detecting in a tissue sample the level of expression of and/or activity of a protein encoded by at least one gene or member of a gene family of Table 1, wherein differential expression or activity of the gene or member of a gene family is indicative of abnormal bone tissue deposition.

6. A method of monitoring the treatment of a patient with a condition characterized by abnormal bone tissue deposition, comprising:
(a) administering a pharmaceutical composition to the patient;
(b) preparing a gene expression profile from a cell or tissue sample from the patient and/or assaying an activity of a protein encoded by at least one gene or a member of a gene family of Table 1; and
(c) comparing the patient expression profile or activity to an expression profile or activity from a precursor stem cell population or an osteoblast cell population.

7. A method of treating a patient with a condition characterized by an abnormal deposition of bone tissue, comprising:
(a) administering to the patient a pharmaceutical composition, wherein the composition alters the expression and/or activity of a protein encoded by at least one gene or member of a gene family in Table 1;
(b) preparing a gene expression profile from and/or assaying an activity in a cell or tissue sample from the patient comprising precursor stem cells; and
(c) comparing the patient expression profile and/or activity to a gene expression profile or activity from an untreated cell population comprising precursor stem cells.

8. A method of diagnosing a condition characterized by an abnormal rate of formation of osteoblasts, comprising detecting in a tissue sample a level of expression of and/or activity of a protein encoded by at least one gene or member of a gene family from Table 1, wherein differential expression and/or activity of the gene or member of a gene family is indicative of an abnormal rate of formation of osteoblasts.

9. A method of monitoring the treatment of a patient with a condition characterized by abnormal rate of formation of osteoblasts, comprising:
(a) administering a pharmaceutical composition to the patient;
(b) preparing a gene expression profile and/or assaying an activity of at least one gene or member of a gene family from Table 1 in a cell or tissue sample from the patient; and
(c) comparing the patient gene expression profile and/or activity to a gene expression profile or activity from a precursor stem cell population or an osteoblast cell population.

10. A method of treating a patient with a condition characterized by an abnormal rate of formation of osteoblasts, comprising:
(a) administering to the patient a pharmaceutical composition, wherein the composition alters the expression and/or activity of at least one gene or member of a gene family in Table 1;
(b) preparing a gene expression profile and/or assaying an activity in a cell or tissue sample from the patient comprising precursor stem cells; and
(c) comparing the patient expression profile and/or activity to a gene expression profile or activity from an untreated cell population comprising precursor stem cells.

11. A method of diagnosing osteoporosis in a patient, comprising detecting the level of expression and/or activity in a tissue sample of at least one gene or member of a gene family from Table 1; wherein differential expression or activity is indicative of osteoporosis.

12. A method of monitoring the treatment of a patient with osteoporosis, comprising:
(a) administering a pharmaceutical composition to the patient;
(b) preparing a gene expression profile and/or assaying an activity of at least one gene or member of a gene family of Table 1 in a cell or tissue sample from the patient; and
(c) comparing the patient gene expression profile and/or activity to a gene expression profile or activity in a precursor stem cell population or an osteoblast cell population.

13. A method of treating a patient with osteoporosis, comprising:
(a) administering to the patient a pharmaceutical composition, wherein the composition alters the expression and/or activity of at least one gene or member of a gene family in Table 1;
(b) preparing a gene expression profile and/or assaying an activity from a cell or tissue sample from the patient comprising precursor stem cells; and
(c) comparing the patient expression profile and/or activity to a gene expression profile or activity from an untreated cell population comprising precursor stem cells.

14. A method of screening for an agent capable of ameliorating the effects of osteoporosis, comprising:
(a) exposing a cell to the agent; and
(b) detecting the expression and/or activity level of one or more-genes or members of a gene family of Table 1.

15. A method of monitoring the progression of bone tissue deposition in a patient, comprising detecting the level of expression and/or activity in a tissue sample of at least one gene or member of a gene family from Table 1; wherein differential expression and/or activity is indicative of bone tissue deposition.

16. A method of screening for an agent capable of modulating the deposition of bone tissue, comprising:
(a) exposing a cell to the agent; and
(b) detecting the expression and/or activity level of at least one gene or member of a gene family of Table 1.

17. The method of any one of claims 1-16, wherein expression and/or activity levels of at least 2 genes are detected

18. The method of any one of claims 1-16, wherein expression and/or activity levels of at least 3 genes are detected.

19. The method of any one of claims 1-16, wherein expression and/or activity levels of at least 4 genes are detected.

20. The method of any one of claims 1-16, wherein expression and/or activity levels of at least 5 genes are detected.

21. The method of any one of claims 1-16, wherein expression and/or activity levels of at least 6 genes are detected.

22. The method of any one of claims 1-16, wherein expression and/or activity levels of at least 7 genes are detected.

23. The method of any one of claims 1-16, wherein expression and/or activity levels of at least 8 genes are detected.

24. The method of any one of claims 1-16, wherein expression and/or activity levels of at least 9 genes are detected.

25. The method of any one of claims 1-16, wherein expression and/or activity levels of at least 10 genes are detected.

26. The method of any one of claims 1-16, wherein expression and/or activity levels of all the genes in Table 1 are detected.

27. A composition comprising at least two oligonucleotides, wherein each of the oligonucleotides comprises a sequence that specifically hybridizes to a gene or member of a gene family of Table 1.

28. A composition according to claim 27, wherein the composition comprises at least 3 oligonucleotides, wherein each of the oligonucleotides comprises a sequence that specifically hybridizes to a gene or member of a gene family of Table 1.

29. A composition according to claim 27, wherein the composition comprises at least 5 oligonucleotides, wherein each of the oligonucleotides comprises a sequence that specifically hybridizes to a gene or member of a gene family of Table 1.

30. A composition according to claim 27, wherein the composition comprises at least 7 oligonucleotides, wherein each of the oligonucleotides comprises a sequence that specifically hybridizes to a gene or member of a gene family of Table 1.

31. A composition according to claim 27, wherein the composition comprises at least 10 oligonucleotides, wherein each of the oligonucleotides comprises a sequence that specifically hybridizes to a gene or member of a gene family of Table 1.

32. A composition according to any one of claims 27-31, wherein the oligonucleotides are attached to a solid support.

33. A composition according to claim 32, wherein the solid support is selected from a group consisting of a membrane, a glass support, a filter, a tissue culture dish, a polymeric material and a silicon support.

34. A solid support to which is attached at least two oligonucleotides, wherein each of the oligonucleotides comprises a sequence that specifically hybridizes to a gene or member of a gene family of Table 1.

35. A solid support according to claim 34, wherein at least one oligonucleotide is attached covalently.

36. A solid support according to claim 34, wherein at least one oligonucleotide is attached non-covalently.

37. A solid support of claim 34, wherein the solid support is an array comprising at least 10 different oligonucleotides in discrete locations per square centimeter.

38. A solid support of claim 34, wherein the array comprises at least 100 different oligonucleotides in discrete locations per square centimeter.

39. A solid support of claim 34, wherein the array comprises at least 1000 different oligonucleotides in discrete locations per square centimeter.

40. A solid support of claim 34, wherein the array comprises at least 10,000 different oligonucleotides in discrete locations per square centimeter.

41. A computer system comprising:
(a) a database containing information identifying the expression and/or activity level in osteoblasts of a set of genes comprising one or more genes or members of a gene family in Table 1; and
(b) a user interface to view the information.

42. A computer system of claim 41, wherein the database further comprises sequence information for the genes or gene families.

43. A computer system of claim 41, wherein the database further comprises information identifying the expression and/or activity level in precursor stem cells of at least one gene or member of a gene family of Table 1.

44. A computer system of claim 41, wherein the database further comprises information identifying the expression level a set of genes indicative of a condition characterized by abnormal bone tissue deposition.

45. A computer system of any of claims 4144, further comprising records including descriptive information from an external database, which information correlates said genes to records in the external database.

46. A computer system of claim 45, wherein the external database is GenBank.

47. A method of using a computer system of any one of claims 41-44 to present information identifying the expression level in a tissue or cell of a set of genes comprising at least two of the genes or members of gene families in Table 1, comprising:
(a) comparing the expression level of at least one gene or member of a gene family in Table 1 in the tissue or cell to the level of expression of the gene in the database.

48. A method of claim 47, wherein the expression levels of at least two genes are compared.

49. A method of claim 47, wherein the expression levels of at least five genes are compared.

50. A method of claim 47, wherein the expression levels of at least ten genes are compared.

51. A method of claim 47, further comprising the step of displaying the level of expression of at lest one gene in the tissue or cell sample compared to the expression level in osteoblasts.
